(19) United States
(12) Patent Application Publication
(10) Pub. No.: US 2006/0029560 A1
(43) Pub. Date: Feb. 9, 2006

(54) Two-coat cosmetic product comprising at least one silicone polymer

(76) Inventor: Xavier Blin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/182,829

(22) Filed: Jul. 18, 2005

Related U.S. Application Data
(60) Provisional application No. 60/591,929, filed on Jul. 29, 2004.

(30) Foreign Application Priority Data
Jul. 16, 2004 (FR) 04 51551

(51) Int. Cl. A61K 8/894

(52) U.S. Cl. 424/70.12

(57) ABSTRACT

The present disclosure relates to a cosmetic product comprising at least one first and at least one second compositions, wherein the at least one first composition comprises a silicone polymer of formula (I) and the at least one second composition comprises a cosmetically acceptable medium. The present disclosure also relates to a makeup process and to a makeup kit comprising the product.

This application claims benefit of U.S. Provisional Application No. 60/591,929, filed Jul. 29, 2004, the contents of which are incorporated herein by reference. This application also claims benefit of priority under 35 U.S.C. § 119 to French Patent Application No. 04 51551, filed Jul. 16, 2004, the contents of which are also incorporated herein by reference.

The present disclosure relates to a cosmetic product comprising at least two compositions that may be applied successively to either facial or body skin, the lower and upper eyelids, the lips and the integuments, for instance the nails, the eyebrows, the eyelashes or the hair. The present disclosure also relates to a body or facial makeup process using these two compositions.

Each composition may be chosen from a foundation, a makeup rouge, an eyeshadow, a concealer product, a blusher, a loose or compacted powder, a lipstick, a lip balm, a lip gloss, a lip pencil, an eye pencil, a mascara, an eyeliner, a nail varnish, a body makeup product or a skin coloring product.

The present disclosure further relates to a composition that has good staying power and is also glossy.

Good staying power can be reflected by, for example, at least one property chosen from good color fastness, such as when the composition comes into contact with water, saliva, sweat, sebum or fatty substances; the absence of transfer of the composition onto objects with which it comes into contact; the absence of migration beyond the initial line of the makeup; and the staying power of the gloss over time.

Poor staying power may be characterized by a modification of the color (color change or fading) generally following interaction with the sebum and/or sweat secreted by the skin, in the case of foundations and makeup rouges, or interaction with saliva in the case of lipsticks. This can oblige the user to freshen the makeup very regularly, which may constitute a wasting of time.

Lip and skin makeup compositions that can transfer little or not at all are compositions that have the advantage of forming a deposit that does not become deposited, at least partially, onto the supports with which they come into contact (e.g., glass, clothing, cigarette or fabrics).

The known transfer-resistant compositions are generally based on silicone resins and on volatile silicone oils, and, although they may have improved staying power properties, they can have the drawback of leaving on the skin and the lips, after evaporation of the volatile silicone oils, a film that may become uncomfortable over time (sensation of drying-out and of tautness), which can put a certain number of users off this type of lipstick.

In addition, these compositions based on volatile silicone oils and silicone resins can lead to matt colored films. However, consumers are nowadays in search of products, such as lip or eyelid coloring products, that are glossy while at the same time having good staying power and being transfer-resistant.

For example, among the prior art, mention may also be made of International Patent Application No. WO-A-97/17057, which describes a method for increasing the staying-power and transfer-resistance properties, which consists in applying two compositions one over the other. The composition to be applied has a global Hildebrand solubility parameter of less than 8.5 (cal/cm³)^{1/2}, and the composition to be applied as top coat should contain oils with a calculated partition coefficient ClogP of at least 13.

U.S. Pat. No. 6,001,374 proposes a multi-coat makeup system, which consists in using a composition comprising an alcohol-soluble and water-insoluble resin, which may be applied as base coat or as top coat, and which can have the advantage of not marking an object placed in contact with the makeup and of being resistant to water and to rubbing, while at the same time having a certain level of gloss. However, this composition comprises a water-soluble alcohol, in particular ethanol, which is a compound that can have an irritant nature, a drying effect on the skin and/or for instance, on the lips, and can be particularly uncomfortable when the skin or the lips are damaged.

International Patent Application No. WO 02/067877 describes a method for improving the aesthetic properties of a transfer-resistant composition, which consists in applying a second composition onto a film of transfer-resistant composition. The second composition is not supposed to interact chemically with the transfer-resistant composition, so as not to impair its cosmetic properties. Certain products described in the application have an unpleasant odour and are tacky. Other products are not glossy enough.

Accordingly, the present disclosure relates to a novel route for formulating a cosmetic product, such as a makeup product, comprising at least two compositions to be applied successively one on the other. This cosmetic product can have at least one property chosen from those of good gloss, staying power and/or comfort properties.

The present disclosure also relates to a cosmetic product, such as a makeup product, which beneficially combines the at least one of the properties of fastness of color, staying power of the gloss over time, transfer resistance, migration resistance, comfort and gloss.

The inventor has found, surprisingly, that these properties can be achieved by combining at least one first composition comprising at least one certain silicone polymer and a second composition comprising at least one cosmetically acceptable medium. The at least one second composition can comprise, for example, at least one oil.

Thus, the compositions according to the present disclosure may in some embodiments make it possible to obtain a very glossy cosmetic result on application and over time, which does not migrate, does not transfer and shows good staying power, while at the same time being comfortable on application and over time.

The properties of color-fastness, transfer resistance and migration resistance, allied with the glossy appearance, make the composition according to the present disclosure a product that is quite suitable for making lip makeup products such as lipsticks and lip glosses, eye makeup products such as mascaras, eyeliners and eyeshadows, and nail or hair makeup products.

The present disclosure thus relates to cosmetic products for application to the skin, such as the skin of the face or the neck, the lips and the eyelids, comprising at least one first and at least one second compositions, the at least one first composition comprising at least one silicone polymer chosen from those of formula (I):
R¹ₐR²_{b}R³_{c}SiO_{(4-a-b-c)/2} (I)
wherein:
a ranges from 1 to 2.5;
b and c, independently of each other, range from 0.001 to 1.5;
R¹, which may be identical or different, is chosen from:
   C₁ to C₃₀ alkyl radicals, optionally substituted with at least one entity chosen from fluorine atoms and amino and/or carboxyl groups,
   aryl and aralkyl radicals, and
   radicals chosen from those of formula (II):
      —C_{d}H_{2d}—O—(C₂H₄O)ₑ(C₃H₆O)_{f}R⁴ (II)
      wherein:
R⁴ is chosen from C₁ to C₃₀ hydrocarbon-based radicals and R⁵—(CO)— radicals in which R⁵ is a C₁ to C₃₀ hydrocarbon-based radical, and
d, e and f are integers such that d ranges from 0 to 15, and e and f, independently of each other, range from 0 to 50,
and combinations thereof,
R is a radical chosen from those of formula (III):
   -Q-O—X (III)
   wherein:
Q is a divalent C₂ to C₂₀ hydrocarbon-based radical which may include at least one ether bond and/or at least one ester bond, and
X is a polyhydroxylated hydrocarbon-based radical,
R³ is chosen from:
   C₁ to C₃₀ alkyl radicals, optionally substituted with at least one entity chosen from fluorine atoms and amino and/or carboxyl groups,
   aryl and aralkyl radicals,
   organosiloxane groups of formula (IV): 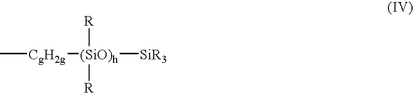
      wherein:
R, which may be identical or different, are chosen from C₁ to C₃₀ alkyl radicals, optionally substituted with at least one entity chosen from fluorine atoms, and aryl and aralkyl radicals,
g and h are integers such that g ranges from 1 to 5 and h ranges from 0 to 500,
   and the second composition, which is different from the first composition, comprising a cosmetically acceptable medium.

The present disclosure also relates to a cosmetic product for application to the integuments, such as the nails, the hair or the eyebrows, comprising at least one first and at least one second compositions, the at least one first composition comprising, in a cosmetically acceptable organic liquid medium, at least one silicone polymer as described above, and the at least one second composition, different from the first composition, comprising a cosmetically acceptable medium.

The product of the present disclosure can be a skin, nail or hair makeup product.

As used herein, the term “makeup product” is understood to mean a product comprising at least one coloring agent allowing the deposition of a color onto a human keratin material (the skin or the integuments) by application to the keratin material of products such as lipsticks, makeup powders, eyeliners, foundations or self-tanning products, or semi-permanent makeup products (tattoos).

The product according to the present disclosure comprises at least two cosmetically acceptable compositions packaged either separately or together, in the same packaging article or in two separate packaging articles.

For example, these compositions may be packaged separately and, in one embodiment, are packaged in separate packaging articles.

The present disclosure relates to for instance, a cosmetic makeup product in the form of a foundation, a makeup rouge, an eyeshadow, a lipstick, a nail varnish, a product having, for example, care properties, a mascara, an eyeliner, a concealer product, a body makeup product (of the tattoo type) or a hair makeup product.

In addition, the present disclosure relates to a makeup kit comprising a cosmetic makeup product as defined above, in which the various compositions are packaged separately and can be, for example, accompanied by suitable applicators. The applicators may be fine brushes, coarse brushes, pens, pencils, felts, quills, sponges, tubes and/or foam tips.

The at least one first composition of the product according to the present disclosure may be a base coat applied to the keratin material, and the at least one second composition a top coat. However, it is possible to apply under the at least one first coat an undercoat that may or may not have the constitution of the second coat.

It is also possible to deposit a top coat onto the at least one second coat that may or may not have a constitution identical to that of the at least one first coat. In one embodiment of the present disclosure, the makeup obtained is a two-coat makeup.

The at least one second composition may also be a base coat applied to the keratin material, and the at least one first composition may be a top coat.

For example, the base coat can be a lipstick, a foundation, a mascara, a lip gloss, an eyeliner, a nail varnish, a nailcare product or a body makeup product, and the top coat can be a care or protective product.

The present disclosure also relates to a process for making up the skin and/or the lips and/or the integuments, which comprises applying to the skin and/or the lips and/or the integuments a cosmetic product as defined above.

The present disclosure still further relates to a cosmetic process for caring for or making up human skin and/or lips and/or integuments, comprising applying to the skin, the lips and/or the integuments at least one first coat of at least one first composition comprising at least one silicone polymer as described above, and then in applying, onto all or part of the at least one first coat, at least one second coat of at least one second composition comprising a cosmetically acceptable medium.

The present disclosure also relates to a cosmetic process for caring for or making up human skin and/or lips and/or integuments, comprising applying to the skin, the lips and/or the integuments at least one first coat of at least one first composition comprising a cosmetically acceptable medium, and then in applying, onto all or part of the at least one first coat, at least one second coat of at least one second composition comprising at least one silicone polymer as described above.

The product according to the present disclosure may be applied to the skin of either the face, the scalp or the body, the lips, the inner edge of the lower eyelids, and integuments, for instance the nails, the eyelashes, the hair, the eyebrows or other bodily hairs. The at least one second composition may form patterns and may be applied with a pen, a pencil or any other instrument (sponge, finger, fine brush, coarse brush or quill). The makeup may also be applied to makeup accessories, for instance false nails, false eyelashes or wigs, or alternatively dots or patches that adhere to the skin or the lips (such as beauty spots).

The present disclosure also relates to the cosmetic use of the cosmetic product defined above to improve the properties of comfort and/or gloss and/or transfer resistance and/or migration resistance and/or color fastness of the makeup, on the skin and/or the lips and/or the integuments.

The present disclosure still further relates to the use of a cosmetic product comprising at least one first and at least one second composition, the at least one first composition comprising a silicone polymer as described above, and the at least one second composition comprising a cosmetically acceptable medium, to give the skin and/or the lips and/or the integuments a comfortable, glossy, transfer-resistant, migration-resistant cosmetic result that shows good color-fastness.

First Composition

The at least one first composition according to the present disclosure comprises at least one silicone polymer comprising at least one polyhydroxylated unit.

When introduced in sufficient amount, the at least one silicone polymer has the benefit of improving the staying power while at the same time maintaining the gloss.

Silicone polymers that may be used in the at least one first composition are described in detail in European Patent Application No. EP 1 213 316, which is incorporated into the present application by reference.

For example, the silicone polymers that may be used in the at least one first composition are chosen from those of formula (I):
R¹ₐR²_{b}R³_{c}SiO_{(4-a-b-c)/2} (I)
wherein:
a ranges from 1 to 2.5; and
b and c, independently of each other, range from 0.001 to 1.5,
R¹, which may be identical or different, is chosen from:
   C₁ to C₃₀ alkyl radicals, optionally substituted with at least one entity chosen from fluorine atoms and amino and/or carboxyl groups,
   aryl and aralkyl radicals, and
   radicals of formula (II):
      —C_{d}H_{2d}—O—(C₂H₄O)ₑ(C₃H₆O)_{f}R⁴ (II)
      wherein:
R⁴ is chosen from C₁ to C₃₀ hydrocarbon-based radicals and R⁵—(CO)— radicals in which R⁵ is a C₁ to C₃₀ hydrocarbon-based radical, and
d, e and f are integers such that d ranges from 0 to 15, and e and f, independently of each other, range from 0 to 50, and
combinations thereof,
R² is a radical chosen from those of formula (III):
   -Q-O—X (III)
   wherein:
Q is a divalent C₂ to C₂₀ hydrocarbon-based radical which may comprise at least one ether bond and/or at least one ester bond, and
X is a polyhydroxylated hydrocarbon-based radical,
R³ is an organosiloxane group of formula (IV): 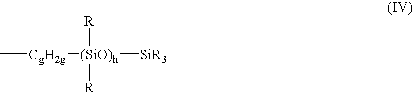
   wherein:
R, which may be identical or different, are chosen from C₁ to C₃₀ alkyl radicals, optionally substituted with at least one entity chosen from fluorine atoms, and aryl and aralkyl radicals,
g and h are integers such that g ranges from 1 to 5 and h ranges from 0 to 500.

When the radicals R are chosen from C₁ to C₃₀ alkyl radicals, optionally substituted with at least one entity chosen from fluorine atoms, aryl radicals and aralkyl radicals, they have the same meaning as the radical R¹ as defined above.

It should be noted that the radicals R¹, R² and R³ of the silicone polymers of general formula (I), as defined above, are randomly distributed, i.e., they appear in the structure of the polymer without any determined order. Similarly, R¹, R² and R³ may feature, respectively, in radicals of different nature in a compound of formula (I).

According to one embodiment of the present disclosure,
a can range from 1.2 to 2.3 and, for example, b and c, independently of each another, can range from 0.05 to 1; and
when R¹ is an alkyl radical, it may be a C₁ to C₃₀ alkyl radical, for instance, a C₁ to C₂₅ alkyl radical, such as a C₁ to C₂₀ alkyl radical, a C₁ to C₁₀ alkyl radical and a C₁ to C₆ alkyl radical, for example, a C₁ to C₄ alkyl radical. For instance, it may be a methyl, ethyl, n-propyl, isopropyl, n-butyl, isobutyl, tert-butyl, pentyl, hexyl, heptyl, octyl, nonyl, decyl, undecyl or lauryl radical. It may also be a cycloalkyl radical such as a cyclopropyl, a cyclobutyl, a cyclopentyl or a cyclohexyl. It may also be a linear or branched monounsaturated or polyunsaturated alkyl radical. It may also be an alkyl radical substituted with at least one fluorine atom, such as trifluoropropyl or heptadecafluorodecyl. It may also be an alkyl radical substituted with at least one amino group, such as 2-aminoethyl, 3-aminopropyl or 3-(2-aminoethyl)aminopropyl. It may also be an alkyl group substituted with at least one carboxyl group, such as 3-carboxypropyl;
R¹ may also be an aryl or aralkyl radical such as a phenyl radical, a tolyl radical, a benzyl radical or a phenethyl radical;
R¹ may also be an organic group chosen from those of formula (II):
   —C_{d}H_{2d}—O—(C₂H₄O)ₑ(C₃H₆O)_{f}R⁴ (II)

According to another embodiment, R¹ may be a hydroxylated radical or a radical derived from the addition reaction of a saturated or unsaturated, linear or branched alkenyl ether, in which d is equal to zero, and thus of formula:
—O—(C₂H₄O)ₑ(C₃H₆O)R⁴

In this case, when e and f are equal to zero, then R¹ is an alkoxy group comprising from 4 to 30 carbon atoms, for example a C₄ to C₁₀ lower alkoxy radical, such as butoxy or pentoxy, or a C₁₁ to C₃₀ higher alkoxy radical, such as oleoxy or stearoxy, namely, for example, cetyl alcohol, oleyl alcohol and stearyl alcohol, or a radical derived from an acid or from a fatty acid, such as acetic acid, lactic acid, butyric acid, oleic acid, stearic acid or behenic acid.

When e and f are greater than 1, then R¹ is a hydroxyl radical derived from the addition reaction of an alkylene oxide.

When e and f are equal to 0, d may be, for example, equal to 3, 5 or 11. In this case, R¹, depending on the nature of the substituent R⁴, may be an allyl ether, pentenyl ether or undecenyl ether radical, or an allyl stearyl ether, pentenyl behenyl ether or undecenyl oleyl ether radical.

When e and f are other than zero, an alkoxy radical and an ester radical are present via a polyoxyalkylene group.

In one embodiment of the present disclosure, irrespective of the nature of e and f, d can be within the range from 3 to 5.

According to another embodiment of the present disclosure, the radical R may be chosen from any one of the radicals defined above, or a combination of two or more of these radicals. For example, R¹ can be an alkyl radical chosen from a methyl radical and a lauryl radical, and combinations thereof.

Moreover, when R¹ represents, in the same compound of formula (I), at least two radicals, for example a methyl radical and a lauryl radical, these radicals appear randomly in the structure, and with a frequency intrinsic thereto.

For example, in one embodiment of the present disclosure, at least 50% of the radicals R¹, for instance at least 70% of the radicals R¹, such as 100% of the radicals R¹, can be methyl radicals.

In another embodiment of the present disclosure,
Q may be, for instance, a divalent hydrocarbon-based radical chosen from:
—(CH₂)₂—, —(CH₂)₃—, —CH₂CH(CH₃)—CH₂, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₂—CH(CH₂CH₂CH₃)—, —CH₂—CH(CH₂CH₃)—, —(CH₂)₃—O—(CH₂)₂—, —(CH₂)₃—O—(CH₂)₂—O—(CH₂)₂—, —(CH₂)₃—O—CH₂CH(CH₃)— and —CH₂—CH(CH₃)—COO(CH₂)₂— radicals.

For example, Q can be a divalent radical chosen from —(CH₂)₂— and —(CH₂)₃—.
X may be a polyhydroxylated hydrocarbon-based radical comprising at least two hydroxyl residues, such as a hydrocarbon-based group chosen from glycerol derivatives and saccharide derivatives.

The glycerol derivatives may be compounds having the following formulae, in which Q has the same meaning as in the formula (III), and s and t are integers ranging from 1 to 20, for instance from 1 to 15, such as from 1 to 10 and from 1 to 5.

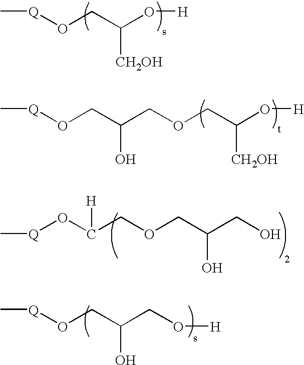

In the above formulae at least one of the hydroxyl groups may be replaced with alkoxy groups or ester groups.

The saccharide derivatives that may be used in the formula (III) can be of monosaccharide type, such as glycosyl, mannosyl, galactosyl, ribosyl, arabinosyl, xylosyl or fructosyl groups, of oligosaccharide type, such as maltosyl, cellobiosyl, lactosyl or maltotriosyl, or of polysaccharide type, such as cellulose or starch.

For example, in one embodiment of the present disclosure, the saccharide groups are of monosaccharide or oligosaccharide type.

In another embodiment of the present disclosure,
each of the radicals R, which may be identical or different, can be chosen from C₁ to C₂₀, for instance, C₁ to C₁₀ and such as C₁ to C₆ alkyl radicals, optionally substituted with at least one fluorine atom. When the radicals R are chosen from the alkyl radicals as defined above, optionally substituted with at least one fluorine atom, they have the same meaning as the radical R¹ as defined above.

According to one embodiment of the present disclosure, g is equal to 2.

According to another embodiment, h ranges from 1 to 50.

In yet another embodiment of the present disclosure, as defined above, the at least one silicone polymer of formula (I) is such that when the radical R² is chosen from those of formula (IIIA):
—C₃H₆O[CH₂CH(OH)CH₂O]ₙH (IIIA)
in which n ranges from 1 to 5, then the radical R¹ is not a C₁₂ alkyl radical.

According to still another embodiment, the at least one silicone polymer of formula (I) that may be used in the present disclosure is such that:
ranges from 1 to 1.4, and b and c, independently of each other, range from 0.02 to 0.03, and
R¹ is a C₁ to C₁₀, for example, C₁ to C₆ and such as C₁ to C₄, alkyl radical,
R² is chosen from the radicals of formula (IIIA):
   —C₃H₆O[CH₂CH(OH)CH₂O]ₙH (IIIA)
   in which:
n ranges from 1 to 5, and
R³ is chosen from the radicals of formula (IVA):
   —C₂H₄(CH₃)₂SiO[(CH₃)₂SiO]ₘSi(CH₃)₃ (IVA)
   in which:
m ranges from 3 to 9.

According to yet another embodiment, the at least one silicone polymer of formula (I), which may be used in the at least one first composition, is such that:
a ranges from 1 to 1.4, and b and c, independently of each other, range from 0.02 to 0.04,
R¹ is a methyl radical,
R² is a radical chosen from those of formula (IIIA) in which n ranges from 1 to 5, and
R³ is a radical chosen from those of formula (IVA) in which m ranges from 3 to 9.

For instance, the at least one silicone polymer of formula (I) used in the at least one first composition may be chosen from polyglyceryl-3 polymethylsiloxyethyl dimethicone, laurylpolyglyceryl-3 polymethylsiloxyethyl dimethicone and polyglyceryl-3 disiloxane dimethicone, the respective formulae of which are:
Polyglyceryl-3 polymethylsiloxyethyl dimethicone (formula (V)): 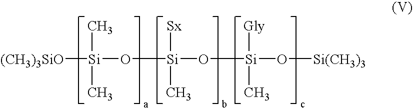
   in which:
Sx is chosen from —C₂H₄[(CH₃)₂SiO]ₘSi(CH₃)₃ radicals
Gly is chosen from —C₃H₆O[CH₂—CH(OH)CH₂O]ₙH radicals
and a ranges from 1 to 1.4, b ranges from 0.02 to 0.04, c ranges from 0.02 to 0.04, m ranges from 3 to 9, and n ranges from 1 to 5
   Polyglyceryl-3 polymethylsiloxyethyl dimethicone (formula (VI)): 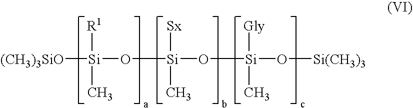
      in which Sx, Gly, a, b, c, m and n have the same meanings as above and R¹ is chosen from methyl radicals and lauryl radicals;
   Polyglyceryl-3 disiloxane dimethicone (formula (VII)): 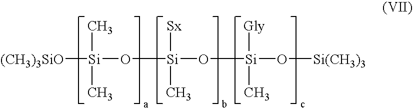
      in which Gly, a, b, c, m and n have the same meanings as above, and
   Sx is chosen from —O(CH₃)₂SiO—Si(CH₃)₃ radicals.

The at least one silicone polymer of formula (I) can be present in the at least one first composition in an amount ranging from 0.1% to 40% by weight, for instance from 0.5% to 30% by weight, such as from 1% to 25% by weight, from 5% to 20% by weight and from 7% to 15% by weight, relative to the total weight of the composition.

According to another embodiment, the at least one silicone polymer of formula (I) can be chosen from the polymers sold by the company Shin-Etsu under the names KF6100®, KF6104® and KF6105®.

According to still another embodiment, the compound designated by the reference KF6104®, sold by the company Shin-Etsu, is suitable for, for instance, preparing a cosmetic product in accordance with the present disclosure that has at least one property chosen from improved staying power and improved mean gloss.

As used herein, the term “staying power” is understood to mean the property of a cosmetic product according to the present disclosure to transfer to a lesser extent onto objects with which it may come into contact, and/or the property of withstanding interaction with liquids, for instance tears or sweat, or contact with foods during a meal in the case of a lipstick, for example, and/or the property of not migrating beyond the initial line of the makeup, for instance in the case of lipsticks, within the wrinkles and fine lines around the lips.

The improvement in the staying power of the product according to the present disclosure may thus be derived from the improvement in at least one of the properties described above. For example, the improvement of the staying power may be derived from the improvement of its transfer resistance and/or from the improvement of its color-fastness.

In one embodiment, the cosmetic product according to the present disclosure can have the benefit of not transferring, at least partially, i.e., of leaving only the merest traces on certain supports with which it may come into contact, such as a glass, a cup, a cigarette, a handkerchief, an item of clothing or the skin. The transfer of a cosmetic composition causes poor staying power of the applied film, making it necessary to regularly freshen the application of the composition.

The cosmetic product according to the present disclosure can also have good color-fastness. The film of product applied to the skin, the lips and/or the integuments can be impaired during contact with liquids, for instance, water or beverages consumed, for example during a meal, or alternatively oils, for instance food oils, or alternatively sebum or saliva. The color-fastness may thus be characterized by the fastness of its color to water and/or the fastness of its color to oil.

Thus, the evaluation of the staying power properties of the cosmetic product according to the present disclosure is characterized by measuring at least one of the following three parameters: the transfer resistance, the color-fastness to water and the color-fastness to oil.

The three parameters can be measured one after the other according to the protocol described below.

The measurements are performed, for example, on the inner face of the forearm, which is first washed and allowed to dry naturally at room temperature for 5 minutes. The at least one first cosmetic composition to be tested, for example a lipstick, is applied to three areas of the inner face of the forearm. The at least one first composition is left to dry for the time required for the volatile solvents that may be present to evaporate off, and the at least one second composition is then applied onto the at least one first composition. The surface area of skin on which the measurements are performed should be greater than at least 1 cm². These measurements are generally taken on circular areas about 3 cm in diameter.

It is necessary for the same amount of cosmetic product to be applied to each of the three areas. This may be checked by measuring the weight of the at least one first and the at least one second cosmetic compositions, after each of the applications, or by preparing beforehand equivalent amounts of sample to be tested. In general, for a surface area of 1 cm², an amount equal to about 2 mg is necessary (if the surface area has a diameter of 3 cm, then an amount of about 28 mg is required).

After applying the cosmetic product, the color, L₁*a₁*b₁*, is measured at each of the three areas, and the mean value obtained corresponds to the initial color of the product. The color measurement may be performed using a Minolta calorimeter of the CR200, CR300, CM500, CM1000 or CM2000 series. In one embodiment of the present disclosure, the Minolta calorimeter of the CR200 series is used.

20 mg/cm² of water are added to each of the three areas to be tested (for surface areas of about 3 cm in diameter, about 280 mg of water should be applied). Each of the areas to be tested is then massaged manually for a few seconds, for example, from 2 to 5 seconds and such as 2 seconds.

A thickness of a commercial white paper handkerchief such as Kleenex, the L₀*a₀*b₀* color of which has been measured, is applied to each made-up area for about 5 seconds and at a force of about 100 g/f, which force may be applied using a digital pressure force gauge DPZ-5N from the constructor Imada Co. Ltd.

The transfer value T is obtained by subtracting the color of the white fabric measured before application on the area to be tested, L₀*a₀*b₀*, and the mean color L₂*a₂*b₂* corresponding to the mean of the values obtained for each handkerchief after applying them to each area of forearm covered with product to be tested.

The color difference ΔE (T) between the color of the handkerchief before and after applying it to the area of the forearm bearing the product is then determined:
ΔE(E)=\sqrt{(L₂*−L₀*)²+(a₂*−a₀*)²+(b₂*−b₀*)²}

The lower the ΔE (T) value obtained, the more the cosmetic product is considered as having a good level of transfer resistance.

In one embodiment, the cosmetic product according to the present disclosure has a transfer value ΔE (T) of less than or equal to 45, for instance less than or equal to 40, such as less than or equal to 35 and ranging from 0 to 45.

The mean color L₃*a₃*b₃* of the product after applying the handkerchief is then measured.

The color-fastness to water may be obtained after performing the transfer test. It is, for example, equal to the color difference between the mean initial color L₁*a₁*b₁* of the product applied to the forearm and the mean color L₃*a₃*b₃* of the area of the forearm bearing the product after applying water and the handkerchief.
ΔE(E)=\sqrt{(L₃*−L₁*)²+(a₃*−a₁*)²+(b₃*−b₁*)²}

The lower the value obtained, the more the cosmetic product is considered as having good color-fastness to water.

According to one embodiment, the cosmetic products according to the present disclosure have a color-fastness to water of less than or equal to 15, for example less than or equal to 10, such as less than or equal to 6. For instance, the color-fastness can range from 0 to 15.

The test of color-fastness to oil is performed by applying to the areas to be tested about 20 mg/cm² of food-type oil onto each area of forearm (such as rapeseed oil, soybean oil or sunflower oil) followed by manual massaging for a few seconds, for instance for 2 to 5 seconds, such as for 2 seconds. Next, a thickness of a commercial white paper handkerchief such as a Kleenex handkerchief is applied to the area for about 5 seconds and at a force of about 100 g/f, which force may be applied using a digital pressure force gauge DPZ-5N from the manufacturer Imada Co. Ltd.

The value of the color fastness to oil, H, is also the difference between the mean color L₄*a₄*b₄* of the product remaining on the forearm after massaging with oil and applying the handkerchief, and the mean color L₁*a₁*b₁* initially measured
ΔE(H)=\sqrt{(L₄*−L₁*)²+(a₄*−a₁*)²+(b₄*−b₁*)²}

The test for the color-fastness to oil is a test that makes it possible to evaluate, for example, the hold of a cosmetic composition such as a lipstick during a meal.

In one embodiment, the cosmetic products according to the present disclosure have a color-fastness to oil of less than or equal to 25, for instance, less than or equal to 10, such as less than or equal to 8. For example, the color fastness to oil can range from 0 to 25.

In another embodiment, the product according to the present disclosure has a transfer value of less than or equal to 35, a color-fastness to water of less than 6 and a color-fastness to oil of less than or equal to 8.

For example, the at least one silicone polymer of formula (I) can be such that when it is in sufficient amount in the at least one first composition, the mean gloss at 600 of a deposit of the product according to the present disclosure, once spread onto a support, is greater than or equal to 30 over 100.

As used herein, the term “mean gloss” is understood to mean the gloss as may be conventionally measured using a gloss meter via the following method.

A. Minolta GM268 gloss meter may be used. The measurements are performed on test areas with a surface area of greater than 0.1 cm. In a conventional manner, the surface areas on which the test product is spread measure about 2.5 cm×4 cm.

The at least one first cosmetic composition to be evaluated is applied to a synthetic surface, of Bioskin type. The amount of cosmetic composition to be applied is about 1 mg/cm². The at least one first composition is left to dry and about 1 mg/cm² of the at least one second composition is then applied.

The gloss value is obtained by measuring the reflectance at an angle of about 600.

Five measurements per sample are necessary, the two measurements having the highest values and the measurement having the lowest value are discarded, and a mean of the remaining three values is taken.

The mean gloss of the cosmetic product according to the present disclosure can be greater than or equal to 30, for instance greater than or equal to 40, such as greater than or equal to 45.

The comfort of the product according to the present disclosure is evaluated according to the test described below. In this test, the comfort is measured by means of a test of pulling a latex strip. This test predicts the capacity of a produced deposit to withstand the flaking and peeling liable to occur following the movements of the skin.

Samples of first cosmetic compositions are applied to surfaces, for example 2.54 cm×2.54 cm, of a strip of latex 2.54 cm wide, obtained, for example, by cutting from the wrist area of a glove of the Ansell Edmond Industrial Technicians type, Ref #390, size 9.

The amount of the at least one first cosmetic composition to be deposited is such that the solids weight of the composition should be about 20 mg.

The at least one first composition is allowed to dry, and the at least one second composition is then applied under the same conditions as above.

The at least one first and at least one second compositions are successively applied to the strip of latex using a disposable lip brush, for example of the type produced by the company Femme Cosmetics, Inc., LA.

The amount of the at least one second cosmetic composition to be applied is such that the solids weight of the composition should be about 20 mg.

The samples thus prepared are left for 24 hours at room temperature.

The weight of the latex strip comprising the deposit of the cosmetic product is then measured (B). Subtraction of the value of the weight of the latex strip free of the cosmetic composition (A) from the value thus measured (B) should correspond to the weight of dry film, and should thus be about 40±2 mg.

The strip of latex bearing the samples of cosmetic composition to be tested is then pulled such that the area supporting the sample should reach a length of 4.445 cm (1.75 inches).

The fragments of film of the cosmetic composition that have detached from the strip of latex are observed, and are then removed by brushing using a lip brush.

The weight of the strip of latex comprising the remaining cosmetic composition is then measured (D).

The percentage weight loss of the film of the cosmetic product is then calculated by means of the following equation:
Comfort index=[(D−A)/(B−A)]×100.

The measurements are repeated three times for each cosmetic product tested. The comfort index of the composition according to the present disclosure is equal to the mean of these three measurements.

For example, the at least one silicone polymer of formula (I) can be such that when it is present in sufficient amount in the cosmetic composition, the comfort index of a deposit of the product, once spread onto a support, is greater than or equal to 80 over 100, such as 90 over 100, and greater than 95 over 100.

The at least one first composition may further comprise, for instance, at least one film-forming agent, which may or may not be combined with at least one auxiliary film-forming agent.

The additional film-forming polymer may be chosen, for example, from one of the polymers described in French Patent Application No. FR 0 450 540, the content of which is incorporated in the present application by reference.

The at least one additional film-forming polymer can be present in the composition according to the present disclosure in an amount ranging from 0.1% to 60% by weight, for instance ranging from 2% to 40% by weight, such as from 5% to 25% by weight, relative to the total weight of the composition.

As used herein, the term “film-forming polymer” is understood to mean a polymer capable of forming, by itself or in the presence of an auxiliary film-forming agent, an insolable and, for instance, continuous film that adheres to a support, such as to keratin materials, for example a cohesive film, such as a film whose cohesion and mechanical properties are such that the film may be isolated from the support.

In one embodiment of the present disclosure, the at least one additional film-forming agent is present in the cosmetic compositions in accordance with the present disclosure in a weight ratio relative to the weight of the at least one silicone polymer of formula (I) of less than 5:1, for instance, less than 1:1, such as less than or equal to 1:2.

Among the film-forming polymers that are suitable for use in the present disclosure, non-limiting mention may be made of acrylic polymers and copolymers, polyurethanes, polyesters, polyamides, polyureas, cellulose-based polymers, for instance nitrocellulose, and silicone polymers.

Silicone resins, which are crosslinked polyorganosiloxane polymers that are generally soluble or swellable in silicone oils, may also be used. Non-limiting examples of these silicone resins that may be mentioned include siloxysilicates, polysilsesquioxanes and polymethylsesquioxanes.

Non-limiting examples of commercially available polymethylsesquioxane resins that may be mentioned include those sold by the company Wacker under the reference Resin MK, such as Belsil PMS MK; those sold by the company Shin-Etsu, for example under the reference KR-220L.

Siloxysilicate resins that may be mentioned, by way of non-limiting example, include the trimethyl siloxysilicate resins sold under the reference SR1000 by the company General Electric, or under the reference TMS803 by the company Wacker. Non-limiting mention may also be made of the trimethyl siloxysilicate resins sold under the name KF-7312J by the company Shin-Etsu, and the resins DC749 and DC593 sold by the company Dow Corning.

Liposoluble silicone polymers that may also be mentioned in a non-limiting manner include silicone polyamides of the polyorganosiloxane type, such as those described in U.S. Pat. Nos. 5,874,069; 5,919,441; 6,051,216; and 5,981,680.

According to one embodiment, the additional film-forming silicone polymer that is suitable for use in the present disclosure may be a copolymer comprising carboxylate groups and polydimethylsiloxane groups.

As disclosed herein, the term “copolymer comprising carboxylate groups and polydimethylsiloxane groups” is understood to mean a copolymer obtained from (a) at least one carboxylic (acid or ester) monomers, and (b) at least one polydimethylsiloxane (PDMS) chains.

As disclosed herein, the term “carboxylic monomer” is understood to mean both carboxylic acid monomers and carboxylic acid ester monomers. Thus, the at least one monomer (a) may be chosen, for example, from acrylic acid, methacrylic acid, maleic acid, fumaric acid, itaconic acid, crotonic acid, and esters thereof. Esters that may be mentioned, by way of non-limiting example, include the following monomers: acrylate, methacrylate, maleate, fumarate, itaconate and/or crotonate. The monomers in ester form can be chosen from linear or branched, such as C₁-C₂₄, for instance C₁-C₂₂ alkyl acrylates and methacrylates, the alkyl radical can be chosen from, for example, methyl, ethyl, stearyl, butyl and 2-ethylhexyl radicals, and mixtures thereof.

The copolymer may comprise as carboxylate groups at least one group chosen from acrylic acid and methacrylic acid, and methyl, ethyl, stearyl, butyl or 2-ethylhexyl acrylate or methacrylate, and mixtures thereof.

As used herein, the term “polydimethylsiloxanes” (also known as organopolysiloxanes and abbreviated as PDMS) is understood to mean, in accordance with what is generally accepted, any organosilicon polymer or oligomer of linear structure, of variable molecular weight, obtained by polymerization and/or polycondensation of suitably functionalized silanes, and comprising a repetition of main units in which the silicon atoms are linked together via oxygen atoms (siloxane bond ≡Si—O—Si≡), comprising trimethyl radicals directly linked via a carbon atom to the silicon atoms. The PDMS chains that may be used to obtain the copolymer comprise at least one polymerizable radical group, for example located on at least one of the ends of the chain, i.e., the PDMS may comprise, for example, a polymerizable radical group on the two ends of the chain or one polymerizable radical group on one end of the chain and one trimethylsilyl end group on the other end of the chain. The polymerizable radical group may be, for instance, an acrylic or methacrylic group, for example, a group CH₂═CR₁—CO—O—R₂, in which R₁ is chosen from a hydrogen atom and methyl groups and R₂ is chosen from —CH₂—, —(CH₂)ₙ— with n=3, 5, 8 or 10, —CH₂—CH(CH₃)—CH₂—, —CH₂—CH₂—O—CH₂—CH₂—, —CH₂—CH₂—O—CH₂—CH₂—CH(CH₃)—CH₂—, and —CH₂—CH₂—O—CH₂ CH₂—O—CH₂—CH₂—CH₂—.

The copolymers used are generally obtained according to the usual methods of polymerization and grafting, for example by free-radical polymerization (A) of a PDMS comprising at least one polymerizable radical group (for example on one of the ends of the chain or on both ends) and (B) of at least one carboxylic monomer, as described, for example, in U.S. Pat. Nos. 5,061,481 and 5,219,560.

The copolymers obtained generally have a molecular weight ranging from 3,000 to 200,000, such as from 5,000 to 100,000.

The copolymer may be in its native form or in dispersed form in a solvent such as lower alcohols comprising from 2 to 8 carbon atoms, for instance isopropyl alcohol, or oils, for instance volatile silicone oils (for example cyclopentasiloxane).

As copolymers that may be used, non-limiting mention may be made, for example, of copolymers of acrylic acid and of stearyl acrylate comprising polydimethylsiloxane grafts, copolymers of stearyl methacrylate comprising polydimethylsiloxane grafts, copolymers of acrylic acid and of stearyl methacrylate comprising polydimethylsiloxane grafts, copolymers of methyl methacrylate, butyl methacrylate, 2-ethylhexylacrylate and stearyl methacrylate comprising polydimethylsiloxane grafts. Among copolymers that may be used, non-limiting mention may be made for example of the copolymers sold by the company Shin-Etsu under the names KP-561 (CTFA name: acrylates/dimethicone), KP-541 in which the copolymer is dispersed at 60% by weight in isopropyl alcohol (CTFA name: acrylates/dimethicone and, isopropyl alcohol), KP-545 in which the copolymer is dispersed at 30% in cyclopentasiloxane (CTFA name: acrylates/dimethicone and cyclopentasiloxane). According to one embodiment of the present disclosure, KP561 is can be used; this copolymer is not dispersed in a solvent, but is in waxy form, its melting point being about 30° C.

Second Composition

The cosmetic product according to the present disclosure comprises a second composition comprising a cosmetically acceptable medium.

As used herein, the term “cosmetically acceptable medium” denotes a non-toxic medium that may be applied to human skin or lips. The physiologically acceptable medium is generally suited to the nature of the support to which the composition is to be applied, and also to the appearance in which the composition is intended to be packaged.

The physiologically acceptable medium may comprise an aqueous phase and/or a fatty phase.

According to one embodiment of the present disclosure, the aqueous phase or the fatty phase may form the continuous phase of the composition.

The at least one second composition can be chosen such that it improves at least one cosmetic property of the at least one first composition, when it is applied alone to the keratin material, without, however, degrading one of the other cosmetic properties of the said cosmetic composition.

According to one embodiment, the at least one second composition can improve the gloss, and also the gloss staying power, of the at least one first composition.

According to another embodiment, the at least one first composition predominantly comprises by weight, or even consists of, hydrocarbon-based compounds, and the at least one second composition predominantly comprises by weight, or even consists of, silicone compounds. The terms “silicone” and “hydrocarbon-based” have the same meaning as previously. As used herein, the term “predominantly comprises” is understood to mean that it comprises at least 50% by weight, for instance at least 60% by weight, such as at least 70% by weight, for example at least 80% by weight, at least 90% by weight, and at least 95% by weight.

According to another embodiment, the at least one first composition predominantly comprises by weight, or even consists of, silicone compounds, and the at least one second composition predominantly comprises by weight, or even consists of, hydrocarbon-based compounds.

According to another embodiment, the at least one second composition is transparent.

As used herein, the term “transparent composition” is understood to mean a composition that is transparent to translucent, i.e., it transmits at least 40% light at a wavelength of 750 nm, for instance at least 50% light. The transmission measurement is performed using a Cary 300 Scan UV-visible spectrophotometer from the company Varian, according to the following protocol:

The composition is poured above its melting point into a spectrophotometer cuvette of square cross-section with a side length of 10 mm.

The sample of the composition is then cooled for 24 hours at 35° C. and then maintained in a chamber thermostatically regulated at 20° C. for 24 hours.

The light transmitted through the sample of the composition is then measured on the spectrophotometer by scanning with wavelengths ranging from 700 nm to 800 nm, the measurement being performed in transmission mode.

The percentage of light transmitted through the sample of the composition at a wavelength of 750 nm is then determined.

When the at least one second composition is transparent, it can comprise, for example, less than 5%, for instance less than 2%, such as less than 1% of pigments.

For example, the at least one first and/or the at least one second compositions may comprise, independently of the other, at least one film-forming agent, which may or may not be combined with at least one auxiliary film-forming agent.

The at least one additional film-forming polymer may be one of the polymers described in French Patent Application FR 0 450 540, the content of which is incorporated in the present application by reference.

The at least one additional film-forming polymer can be present in the at least one first or at least one second composition according to the present disclosure in an amount ranging from 0.1% to 60% by weight, for instance ranging from 2% to 40% by weight, such as from 5% to 25% by weight, relative to the total weight of the composition.

For example, the at least one film-forming agent may be present in the at least one first or at least one second composition in a weight ratio relative to the weight of the at least one silicone polymer of formula (I) of less than 5:1, for instance less than 1:1, such as less than or equal to 1:2.

Among the film-forming polymers that are suitable for use in the present disclosure, non-limiting mention may be made of acrylic polymers and copolymers, polyurethanes, polyesters, polyamides, polyureas, cellulose-based polymers, for instance nitrocellulose, and silicone polymers.

It is also possible, for example, to use silicone resins, which are crosslinked polyorganosiloxane polymers that are generally soluble or swellable in silicone oils.

Non-limiting examples of these silicone resins that may be mentioned include siloxysilicates, polysilsesquioxanes and polymethylsesquioxanes.

Non-limiting examples of commercially available polymethylsesquioxane resins that may be mentioned include those sold by the company Wacker under the name Resin MK, such as Belsil PMS MK; those sold by the company Shin-Etsu, for example under the name KR-220L.

Siloxysilicate resins that may also be mentioned in a non-limiting manner include the trimethyl siloxysilicate resins sold under the name SR1000 by the company General Electric, or under the name TMS803 by the company Wacker. Non-limiting mention may also be made of the trimethyl siloxysilicate resins sold under the name KF-7312J by the company Shin-Etsu, and the resins DC749 and DC593 sold by the company Dow Corning.

Liposoluble silicone polymers that may also be mentioned in a non-limiting manner include silicone polyamides of the polyorganosiloxane type, such as those described in U.S. Pat. Nos. 5,874,069; 5,919,441; 6,051,216; and 5,981,680.

According to one embodiment, a film-forming silicone polymer that is suitable for use in the present disclosure can be a copolymer comprising carboxylate groups and polydimethylsiloxane groups.

The expression “copolymer comprising carboxylate groups and polydimethylsiloxane groups” is understood to mean a copolymer obtained from (a) at least one carboxylic (acid or ester) monomer, and (b) at least one polydimethylsiloxane (PDMS) chain.

The term “carboxylic monomer” is understood to mean both carboxylic acid monomers and carboxylic acid ester monomers. Thus, the at least one monomer (a) may be chosen, for example, from acrylic acid, methacrylic acid, maleic acid, fumaric acid, itaconic acid, crotonic acid, and esters thereof. Esters that may be mentioned, in a non-limiting manner, include the following monomers: acrylate, methacrylate, maleate, fumarate, itaconate and/or crotonate. The monomers in ester form can be, for example, chosen from linear or branched, such as C₁-C₂₄ and C₁-C₂₂ alkyl acrylates and methacrylates, wherein the alkyl radical can be chosen from, for example, methyl, ethyl, stearyl, butyl and 2-ethylhexyl radicals, and mixtures thereof.

The at least one copolymer may comprise as carboxylate groups at least one group chosen from acrylic acid and methacrylic acid, and methyl, ethyl, stearyl, butyl or 2-ethylhexyl acrylate or methacrylate, and mixtures thereof.

The term “polydimethylsiloxanes” (also known as organopolysiloxanes and abbreviated as PDMS) is understood to mean, in accordance with what is generally accepted, any organosilicon polymer or oligomer of linear structure, of variable molecular weight, obtained by polymerization and/or polycondensation of suitably functionalized silanes, and comprising a repetition of main units in which the silicon atoms are linked together via oxygen atoms (siloxane bond ≡Si—O—Si≡), comprising trimethyl radicals directly linked via a carbon atom to the silicon atoms. The PDMS chains that may be used to obtain the copolymer comprise at least one polymerizable radical group, for example, located on at least one of the ends of the chain, i.e., the PDMS may comprise, for example, a polymerizable radical group on the two ends of the chain or one polymerizable radical group on one end of the chain and one trimethylsilyl end group on the other end of the chain. The polymerizable radical group can be chosen from, for instance, an acrylic or methacrylic group, such as a group CH₂═CR₁—CO—O—R₂, in which R₁ is chosen from a hydrogen atom and methyl groups and R₂ is chosen from —CH₂—, —(CH₂)ₙ— with n=3, 5, 8 or 10, —CH₂—CH(CH₃)—CH₂—, —CH₂—CH₂—O—CH₂—CH₂—, —CH₂—CH₂—O—CH₂—CH₂—CH(CH₃)—CH₂—, and —CH₂—CH₂—O—CH₂ CH₂—O—CH₂—CH₂—CH₂— radicals.

The copolymers used can be obtained according to the usual methods of polymerization and grafting, for example by free-radical polymerization (A) of a PDMS comprising at least one polymerizable radical group (for example on one of the ends of the chain or on both ends) and (B) of at least one carboxylic monomer, as described, for example, in U.S. Pat. Nos. 5,061,481 and 5,219,560.

The copolymers obtained can have a molecular weight ranging from 3,000 to 200,000, such as from 5,000 to 100,000.

The copolymer may be in its native form or in dispersed form in a solvent such as lower alcohols comprising from 2 to 8 carbon atoms, for instance isopropyl alcohol, or oils, for instance volatile silicone oils (for example cyclopentasiloxane).

Among the copolymers that may be used, non-limiting mention may be made, for example, of copolymers of acrylic acid and of stearyl acrylate comprising polydimethylsiloxane grafts, copolymers of stearyl methacrylate comprising polydimethylsiloxane grafts, copolymers of acrylic acid and of stearyl methacrylate comprising polydimethylsiloxane grafts, copolymers of methyl methacrylate, butyl methacrylate, 2-ethylhexylacrylate and stearyl methacrylate comprising polydimethylsiloxane grafts. Among copolymers that may be used, non-limiting mention may be made of, for example, the copolymers sold by the company Shin-Etsu under the names KP-561 (CTFA name: acrylates/dimethicone), KP-541 in which the copolymer is dispersed at 60% by weight in isopropyl alcohol (CTFA name: acrylates/dimethicone and isopropyl alcohol), KP-545 in which the copolymer is dispersed at 30% in cyclopentasiloxane (CTFA name: acrylates/dimethicone and cyclopentasiloxane). According to one embodiment of the present disclosure, KP561 is used; this copolymer is not dispersed in a solvent, but is in waxy form, its melting point being about 30° C.

The at least one first and at least one second compositions may comprise, independently of each other, a fatty phase, comprising, for instance, fatty substances that are liquid at room temperature (20-25° C.) and atmospheric pressure (oils) and/or waxes or pasty fatty substances.

The at least one oil may be present in an amount, when present, ranging from 0.1% to 99% by weight, for instance, ranging from 1% to 90% by weight, for example, from 5% to 70% by weight, such as from 10% to 60% by weight and from 20% to 50% by weight, relative to the total weight of the at least one first or at least one second cosmetic composition.

The oils that are suitable for preparing the at least one first or at least one second cosmetic compositions may be volatile or non-volatile, silicone or non-silicone oils.

The volatile or non-volatile oils may be hydrocarbon-based oils, for instance of plant or animal origin, synthetic oils, silicone oils, fluoro oils or mixtures thereof. As used herein, the term “hydrocarbon-based oil” is understood to mean an oil mainly comprising hydrogen and carbon atoms and possibly oxygen, nitrogen, sulfur and/or phosphorus atoms.

The volatile hydrocarbon-based oils may be chosen from hydrocarbon-based oils comprising from 8 to 16 carbon atoms, for example, branched C₈-C₁₆ alkanes (also known as isoparaffins), for instance isododecane (also known as 2,2,4,4,6pentamethylheptane), isodecane, isohexadecane, and for example the oils sold under the trade names Isopar® or Permethyl®.

Volatile oils that may also be used include volatile silicones, for instance volatile linear or cyclic silicone oils, such as those with a viscosity less than or equal to 8 centistokes (8×10⁻⁶ m²/s) and for instance comprising from 2 to 10 silicon atoms, such as from 2 to 7 silicon atoms, these silicones optionally comprising alkyl or alkoxy groups comprising from 1 to 10 carbon atoms. Among the volatile silicone oils that may be used in the present disclosure, non-limiting mention may be made of dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

It is also possible to use volatile fluorinated solvents such as nonafluoromethoxybutane or perfluoromethylcyclopentane, and mixtures thereof.

The at least one first or at least one second cosmetic compositions according to the present disclosure may also comprise, independently of each other, at least one volatile oil.

According to one embodiment, the at least one first or at least one second cosmetic composition according to the present disclosure comprises less than 30% by weight, such as less than 15% by weight, for instance less than 10% by weight and less than 5% by weight of volatile oil, relative to the total weight of the composition.

According to another embodiment, the at least one first or at least one second cosmetic composition is free of volatile oils.

The at least one first or at least one second cosmetic composition may also comprise at least one non-volatile oil.

The non-volatile oils may be chosen for example, from non-volatile hydrocarbon-based oils, which may be fluorinated, and/or non-volatile silicone oils.

Non-volatile hydrocarbon-based oils that may be mentioned in a non-limiting manner include:
hydrocarbon-based oils of animal origin,
hydrocarbon-based oils of plant origin, such as phytostearyl esters, such as phytostearyl oleate, phytostearyl isostearate and lauroyl/octyldodecyl/phytostearyl glutanate (Ajinomoto, Eldew PS203), triglycerides comprising fatty acid esters and glycerol, the fatty acids of which may have varied chain lengths from C₄ to C₂₄, these chains possibly being linear or branched, and saturated or unsaturated; these oils can be, for instance, heptanoic or octanoic triglycerides, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stearineries Dubois or those sold under the names Miglyol 810®, 812® and 818® by the company Dynamit Nobel,
synthetic ethers comprising from 10 to 40 carbon atoms,
linear or branched hydrocarbons of mineral or synthetic origin, such as petroleum jelly, polydecenes, hydrogenated polyisobutene such as Parleam, and squalane, and mixtures thereof, and for example hydrogenated polyisobutene,
short hydrocarbon-based esters.

As used herein, the term “short hydrocarbon-based ester” is understood to mean a hydrocarbon-based ester comprising less than 40 carbon atoms.

The at least one first or at least one second cosmetic composition may comprise, independently of each other, at least one short hydrocarbon-based ester.

The esters in accordance with the present disclosure can be monoesters, diesters or polyesters, for example monoesters, i.e., bearing only one ester functional group. These esters can be linear, branched or cyclic, and saturated or unsaturated. For instance, they can be branched and saturated. They can also be volatile or non-volatile.

For example, the hydrocarbon-based esters may correspond to the formula RCOOR′, in which RCOO is chosen from fatty acid residues comprising from 2 to 28 carbon atoms and R′ is chosen from hydrocarbon-based chains comprising from 1 to 28 carbon atoms. For instance, the groups R and R′ can be such that the corresponding ester is non-volatile.

The monoester, diester or polyester hydrocarbon-based esters, which may be used in the at least one first or at least one second cosmetic compositions, for example, may comprise from 10 to 40 carbon atoms.

These non-volatile esters may be, for instance of C₁₀ to C₂₅ such as C₁₄ to C₂₂. They may be chosen from esters of C₂ to C₁₈ acids, for example C₂ to C₂₀ alcohols or of C₂ to C₈ polyols, or mixtures thereof.

In one embodiment, the non-volatile esters comprise less than 22 carbon atoms.

According to another embodiment, when the hydrocarbon-based ester comprises less than 22 carbon atoms, it is not a volatile oil.

Thus, the esters may be chosen from a non-limiting list comprising neopentanoic acid esters, for instance isodecyl neopentanoate, isotridecyl neopentanoate, isostearyl neopentanoate, octyldodecyl neopentanoate, isononanoic acid esters, for instance isononyl isononanoate, octyl isononanoate, isodecyl isononanoate, isotridecyl isononanoate or isostearyl isononanoate, but also isopropyl alcohol esters, such as isopropyl myristate, isopropyl palmitate, isopropyl stearate or isostearate, cetyl octanoate, tridecyl octanoate, 2-ethylhexyl 4-diheptanoate and palmitate, alkyl benzoate, polyethylene glycol diheptanoate, propylene glycol 2-diethylhexanoate, and mixtures thereof. The ester may also be chosen from synthetic esters, such as of fatty acid, for instance purcellin oil, isopropyl myristate, ethyl palmitate or octyl stearate; hydroxylated esters, for instance isostearyl lactate, octyl hydroxystearate or diisopropyl adipate, and fatty alkyl heptanoates, octanoates or decanoates, and mixtures thereof.

Further non-limiting mention may be made of Isononyl isononanoate, isotridecyl isononanoate and stearyl heptanoate, and mixtures thereof.

According to one embodiment, the short hydrocarbon-based ester used in the first cosmetic composition in accordance with the present disclosure is a mixture of isononyl isononanoate, isotridecyl isononanoate and stearyl heptanoate. The at least one hydrocarbon-based ester may be used in the at least one first or at least one second composition in an amount ranging from 5% to 90%, for instance, from 10% to 60%, such as from 20% to 50% by weight, relative to the total weight of the composition.
polyol esters and pentaerythritol esters, for instance dipentaerythrityl tetrahydroxystearate/tetraisostearate,
esters of diol dimers and of diacid dimers such as the products Lusplan DD-DA5 and DD-DA7 described in French Patent Application No. FR 03/02809, the content of which is incorporated into the present application by reference;
fatty alcohols that are liquid at room temperature, comprising a branched and/or unsaturated carbon-based chain of from 12 to 26 carbon atoms, for instance 2-octyldodecanol, isostearyl alcohol, oleyl alcohol, 2-hexyldecanol, 2-butyloctanol and 2-undecylpentadecanol,
higher fatty acids such as oleic acid, linoleic acid and linolenic acid, and mixtures thereof, and
dialkyl carbonates, the two alkyl chains possibly being identical or different, such as the dicaprylyl carbonate sold under the name Cetiol CC® by Cognis.

The non-volatile silicone oils that may be used in the at least one first or at least one second composition may be non-volatile polydimethylsiloxanes (PDMS), for instance simethicone, polydimethylsiloxanes comprising alkyl or alkoxy groups that are pendent and/or at the end of a silicone chain, these groups each comprising from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and dimethicones or phenyl trimethicones with a viscosity of less than or equal to 100 cSt, and mixtures thereof.

For example, the at least one non-volatile hydrocarbon-based oil present in the at least one first cosmetic composition may be chosen from hydrogenated polyisobutene, isostearyl heptanoate, isononyl isononanoate, isotridecyl isononanoate, diisostearyl malate, dipentaerythrityl tetrahydroxystearate/tetraisostearate, and 2-octyldodecanol.

According to one embodiment, the at least one non-volatile oil present in the at least one first composition is a mixture of hydrogenated polyisobutene, isostearyl heptanoate, isononyl isononanoate, isotridecyl isononanoate, diisostearyl malate, dipentaerythrityl tetrahydroxystearate/tetraisostearate and 2-octyldodecanol.

The non-volatile oils may be present in the at least one first or at least one second composition in an amount, when present, ranging from 20% to 99% by weight, for instance, from 30% to 80% by weight, such as from 40% to 80% by weight, relative to the total weight of the composition.

According to one embodiment, the at least one first or at least one second composition comprises at least one silicone oil, present in an amount ranging from 0% to 90% by weight, for instance from 0.1% to 80% by weight, such as from 2% to 80% by weight, relative to the total weight of the composition.

According to another embodiment, the at least one silicone oil is present in the at least one first composition in a weight ratio, relative to the at least one silicone polymer of formula (I), ranging from 80:1, fro instance 60:1, such as 40:1.

The at least one first or at least one second cosmetic composition may also comprise at least one ester of saccharide and of a carboxylic acid, for instance:
C8 to C22 fatty acid esters of sucrose, such as those described in U.S. Pat. No. 6,238,678 and International Patent Application No. WO 00/10523, the content of which is incorporated into the present application by reference,
C1 to C6 acid esters of sucrose, for instance the sucrose acetate isobutyrate sold under the name SAIB by Eastman SAIB.

The liquid fatty phase of the at least one first or at least one second composition may, where appropriate, be thickened, gelled or structured by incorporating therein a fatty-phase gelling agent, for instance a gelling agent described in the International Patent Application WO 2004/28486, the content of which is incorporated in the present application by reference.

The at least one first or at least one second composition according to the present disclosure may comprise at least one aqueous medium, constituting an aqueous phase, which may form the continuous phase of the composition.

The aqueous phase may comprise water. It may also comprise a mixture of water and of at least one water-miscible organic solvent (miscibility in water of greater than 50% by weight at 25° C.), for instance lower monoalcohols comprising from 1 to 5 carbon atoms, such as ethanol or isopropanol, glycols comprising from 2 to 8 carbon atoms, such as propylene glycol, ethylene glycol, 1,3-butylene glycol or dipropylene glycol, C₃-C₄ ketones and C₂-C₄ aldehydes.

The aqueous phase (water and optionally the at least one water-miscible organic solvent) may be present in an amount ranging from 0.1% to 40% by weight, for instance, ranging from 0.1% to 20% by weight, such as from 0.1% to 10% by weight, relative to the total weight of the composition.

This aqueous phase may, where appropriate, be thickened, gelled or structured by also incorporating therein a conventional aqueous gelling agent, such as of mineral origin, for instance clay, and/or of organic origin, for instance an aqueous gelling polymer.

When the at least one first or at least one second composition is intended to be applied to the nails, the composition can comprise a solvent chosen from:
ketones that are liquid at room temperature, such as methyl ethyl ketone, methyl isobutyl ketone, diisobutyl ketone, isophorone, cyclohexanone or acetone;
alcohols that are liquid at room temperature, such as ethanol, isopropanol, diacetone alcohol, 2-butoxyethanol or cyclohexanol;
glycols that are liquid at room temperature, such as ethylene glycol, propylene glycol, pentylene glycol or glycerol;
propylene glycol ethers that are liquid at room temperature, such as propylene glycol monomethyl ether, propylene glycol monomethyl ether acetate or dipropylene glycol mono-n-butyl ether;
cyclic ethers such as γ-butyrolactone;
short-chain esters (comprising from 3 to 8 carbon atoms in total) such as ethyl acetate, methyl acetate, propyl acetate, isopropyl acetate, n-butyl acetate, isopentyl acetate, methoxypropyl acetate or butyl lactate;
ethers that are liquid at room temperature, such as diethyl ether, dimethyl ether or dichlorodiethyl ether;
alkanes that are liquid at room temperature, such as decane, heptane, dodecane or cyclohexane;
alkyl sulfoxides such as dimethyl sulfoxide;
aldehydes that are liquid at room temperature, such as benzaldehyde or acetaldehyde;
heterocyclic compounds such as tetrahydrofuran;
propylene carbonate or ethyl 3-ethoxypropionate; and
mixtures thereof.

By way of non-limiting example, methyl acetate, isopropyl acetate, methoxypropyl acetate, butyl lactate, acetone, methyl ethyl ketone, diacetone alcohol, γ-butyrolactone, tetrahydrofuran, propylene carbonate, ethyl 3-ethoxypropionate and dimethyl sulfoxide, and mixtures thereof, may be used.

According to one embodiment, the at least one first and at least one second cosmetic compositions in accordance with the present disclosure may be in the form of an emulsion, in which the at least one silicone polymer of formula (I) as defined above may serve as surfactant.

As used herein, the term “emulsion” is understood to mean a system of two immiscible liquids, one of which is finely divided as droplets in the other. The dispersed phase is also known as the “inner or discontinuous phase.” The dispersing phase is also known as the “outer or continuous phase.”

The emulsions in which the dispersed phase is lipophilic, for example plant oil or mineral oil, and the dispersing phase is hydrophilic, for example water, are said to be of aqueous type (O/W: oil-in-water). The emulsions in which the dispersed phase is hydrophilic and the dispersing phase is lipophilic are said to be of oily type (W/O: water-in-oil).

Emulsions known as multiple emulsions, for example W/O/W: water-in-oil-in-water, also exist.

For the purposes of the present disclosure, the emulsions comprise a lipophilic phase and a hydrophilic phase, wherein the latter phase is not water.

Thus, the at least one first or second composition may be in anhydrous emulsion form.

For example, the at least one first or at least one second composition may have, for example, a continuous fatty phase, which may comprise less than 10% by weight of water, for instance, less than 5% by weight of water, such as less than 1% by weight of water, relative to the total weight of the composition.

The at least one first and at least one second compositions may be, for example, anhydrous, i.e., they may comprise less than 5%, for instance less than 3%, such as less than 2% and less than 1% of water relative to the total weight of the composition. They may be, for example, in the form of oily gels, oily liquids, pastes or sticks or alternatively in the form of a vesicular dispersion comprising ionic and/or nonionic liquids.

The at least one first and/or at least one second composition may also comprise at least one wax or at least one pasty fatty substance. These fatty substances may be of animal, plant, mineral or synthetic origin.

As used herein, the term “pasty fatty substance” is understood to mean a lipophilic compound comprising, at a temperature of 23° C., a liquid fraction and a solid fraction. The term “pasty fatty substance” is also intended to include polyvinyl laurate.

As used herein, the term “wax” is understood to mean a lipophilic compound that is solid at room temperature (25° C.), with a reversible solid/liquid change of state, having a melting point of greater than or equal to 30° C., which may be up to 120° C.

The melting point of the wax may be measured using a differential scanning calorimeter (DSC), for example the calorimeter sold under the name DSC30 by the company Mettler.

The waxes may be hydrocarbon-based waxes, fluoro waxes and/or silicone waxes and may be of plant, mineral, animal and/or synthetic origin. For example, the waxes can have a melting point of greater than 25° C., such as greater than or equal to 45° C.

As a wax that may be used in the at least one first or at least one second composition, non-limiting mention may be made of linear hydrocarbon-based waxes. Their melting point can be, for instance, greater than 35° C., for example greater than 55° C., such as greater than 80° C.

The linear hydrocarbon-based waxes can be chosen from, for example, substituted linear alkanes, unsubstituted linear alkanes, unsubstituted linear alkenes and substituted linear alkenes, an unsubstituted compound being composed solely of carbon and hydrogen. The substituents mentioned above do not contain any carbon atoms.

The linear hydrocarbon-based waxes include ethylene polymers and copolymers with a molecular weight ranging from 400 to 800, for example Polywax 500 or Polywax 400 sold by New Phase Technologies.

The linear hydrocarbon-based waxes include linear paraffin waxes, for instance the paraffins S&P 206, S&P 173 and S&P 434 from Strahl & Pitch.

The linear hydrocarbon-based waxes include linear long-chain alcohols, for instance products comprising a mixture of polyethylene and of alcohols, comprising 20 to 50 carbon atoms, such as Performacol 425 or Performacol 550 (mixture in 20/80 proportions) sold by New Phase Technologies.

Non-limiting examples of silicone waxes include:
C20-24 alkyl methicone, C24-28 alkyl dimethicone, C20-24 alkyl dimethicone and C24-28 alkyl dimethicone sold by Archimica Fine Chemicals under the reference SilCare 41M40, SilCare 41M50, SilCare 41M70 and SilCare 41M80,
stearyl dimethicones of reference SilCare 41M65 sold by Archimica or of reference DC-2503 sold by Dow Corning,
the stearoxytrimethylsilanes sold under the reference SilCare 1M71 or DC-580,
the products Abil Wax 9810, 9800 or 2440 from Wacker-Chemie GmbH,
the C30-45 alkyl methicones sold by Dow Corning under the reference AMS-C30 Wax, and also the C30-45 alkyl dimethicones sold under the reference SF1642 or SF1632 by General Electric.

The nature and amount of these fatty substances depend on the desired mechanical properties and textures of the compositions. As a guide, the at least one fatty substance may be present in the at least one first or at least one second composition in an amount, when present, ranging from 0.1% to 50% by weight, such as from 1% to 30% by weight of waxes, relative to the total weight of the composition.

The at least one first and at least one second composition may also comprise at least one additional silicone polymer other than the at least one silicone polymer described above, with a molecular mass of greater than or equal to 200,000 g/mol, for instance ranging from 200,000 to 2,500,000, such as 200,000 to 2,000,000 g/mol. This silicone polymer is also referred to as a high molecular weight silicone polymer.

The viscosity of this additional silicone polymer can range from 100,000 to 5,000,000 cSt, for instance from 100,000 to 1,000,000 cSt, such as from 300,000 to 700,000 cSt, measured according to ASTM standard D-445.

The additional silicone polymer can be chosen from, for example, dimethiconols, fluorosilicones and dimethicones, and mixtures thereof.

The high molecular weight silicone polymer may be the dimethiconol sold by Dow Corning in a polydimethylsiloxane (5 cSt) under the reference D2-9085, the viscosity of the mixture being equal to 1,550 cSt, or the dimethiconol sold by Dow Corning in octamethylcyclotetrasiloxane under the reference DC 1503. The dimethiconol (of molecular weight equal to 1,770,000) sold by Dow Corning under the reference Q2-1403 or Q2-1401 may also be used, the viscosity of the mixture being equal to 4,000 cSt.

The high molecular weight dimethicones according to the present disclosure include the dimethicones described in U.S. Pat. No. 4,152,416. They are sold, for example, under the references SE30, SE33, SE 54 and SE 76.

The at least one first and/or at least one second composition according to the present disclosure may also comprise at least one dyestuff chosen from water-soluble dyes, and pulverulent dyestuffs, for instance pigments, nacres and flakes that are well known to those skilled in the art. The at least one dyestuff may be present, in the at least one first or at least one second composition, in an amount ranging from 0.01% to 50% by weight, such as from 0.01% to 30% by weight, relative to the weight of the composition.

As used herein, the term “pigments” should be understood as meaning white or colored, mineral or organic particles, which are insoluble in the liquid organic phase, and which are intended to color and/or opacify the composition.

As used herein, the term “nacres” should be understood as meaning iridescent particles, such as those produced by certain molluscs in their shell or else synthesized, which are insoluble in the medium of the composition.

As used herein, the term “dyes” should be understood as meaning generally organic compounds that are soluble in fatty substances, for instance oils, or soluble in an aqueous-alcoholic phase.

The at least one first and/or at least one second composition may comprise a pigment dispersant.

The dispersant serves to protect the dispersed particles against aggregation or flocculation. This dispersant may be a surfactant, an oligomer, a polymer or a mixture of several thereof, bearing at least one functional groups having strong affinity for the surface of the particles to be dispersed. For example, they can physically or chemically attach to the surface of the pigments. These dispersants also have at least one functional group that is compatible or soluble in the continuous medium. For instance, 12-hydroxystearic acid esters, and C₈ to C₂₀ fatty acid esters of polyols such as glycerol or diglycerol are used, such as poly(12-hydroxystearic acid) stearate with a molecular weight of about 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As another dispersant that may be used in the at least one first or at least one second composition, non-limiting mention may be made of quaternary ammonium derivatives of polycondensed fatty acids, for instance Solsperse 17,000 sold by the company Avecia, and polydimethylsiloxane/oxypropylene mixtures such as those sold by the company Dow Corning under the references DC2-5185 and DC2-5225 C.

The polydihydroxystearic acid and the 12-hydroxystearic acid esters can be useful in a hydrocarbon-based or fluorinated medium, whereas the mixtures of oxyethylene/oxypropylene dimethylsiloxane can be useful in a silicone medium.

The pigments may be white or colored, and mineral and/or organic. Among the mineral pigments that may be mentioned in a non-limiting manner, are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder.

Among the organic pigments that may be mentioned in a non-limiting manner are carbon black, pigments of D & C type, and lakes based on cochineal carmine or on barium, strontium, calcium or aluminium.

Non-limiting mention may also be made of pigments with an effect such as particles comprising a natural or synthetic, organic or mineral substrate, for example glass, acrylic resins, polyester, polyurethane, polyethylene terephthalate, ceramics or aluminas, the said substrate possibly being coated with metallic substances, for instance aluminium, gold, silver, platinum, copper, bronze or metal oxides, for instance titanium dioxide, iron oxide or chromium oxide, and mixtures thereof.

The nacreous pigments may be chosen from mica coated with titanium or with bismuth oxychloride, titanium mica coated with iron oxides, titanium mica coated especially with ferric blue or with chromium oxide, titanium mica coated with an organic pigment of the abovementioned type and also nacreous pigments based on bismuth oxychloride. Interference pigments, for example comprising liquid crystals or multilayers, may also be used.

The water-soluble dyes can be chosen from, for example, beetroot juice or methylene blue.

The at least one first and/or at least one second cosmetic composition in accordance with the preserit disclosure may also comprise, for example, at least one filler of organic or mineral nature, which for instance, allow them to impart improved stability with regard to exudation.

As used herein, the term “filler” should be understood as meaning colorless or white, mineral or organic solid particles of any form, which are in an insoluble and dispersed form in the medium of the composition, and which are intended to give body or rigidity to the composition, and/or softness, a matt effect and uniformity to the makeup.

The at least one filler used in the at least one first or at least one second composition may be of lamellar, globular, spherical or fibrous form or in any other form intermediate between these defined forms.

The fillers according to the present disclosure may or may not be surface-coated, for example surface-treated with silicones, amino acids, fluoro derivatives or any other substance that promotes the dispersion and compatibility of the filler in the composition.

As used herein, the terms “mineral fillers” and “inorganic fillers” are used interchangeably.

Among the fillers that may be used, non-limiting mention may be made of talc, mica, silica, trimethyl siloxysilicate, kaolin, bentone, precipitated calcium carbonate, magnesium carbonate, magnesium hydrocarbonate, hydroxyapatite, boron nitride, hollow silica microspheres (Silica Beads from Maprecos), glass or ceramic microcapsules, silica-based fillers, for instance Aerosil 200 and Aerosil 300; Sunsphere L-31 and Sunsphere H-31 sold by Asahi Glass; Chemicelen sold by Asahi Chemical; and composites of silica and of titanium dioxide, for instance the TSG series sold by Nippon Sheet Glass, and mixtures thereof.

Among the fillers, non-limiting mention may also be made of polyamide powder (Nylon® Orgasol from Atochem), poly-b-alanine powder and polyethylene powder, polytetrafluoroethylene (Teflon®) powders, lauroyllysine, starch, powders of tetrafluoroethylene polymers, hollow polymer microspheres such as Expancel (Nobel Industrie), precipitated calcium carbonate, magnesium carbonate, magnesium hydrocarbonate, metal soaps derived from organic carboxylic acids comprising from 8 to 22 carbon atoms, such as from 12 to 18 carbon atoms, for example zinc stearate, magnesium stearate or lithium stearate, zinc laurate or magnesium myristate, and Polypore® L 200 (Chemdal Corporation), silicone resin microbeads (for example Tospearl® from Toshiba), polyurethane powders, for instance powders of crosslinked polyurethane comprising a copolymer, the copolymer comprising trimethylol hexyllactone. For example, it may be a hexamethylene diisocyanate/trimethylol hexyllactone polymer. Such particles are especially commercially available, for example, under the name Plastic Powder D-400® or Plastic Powder D-800® from the company Toshiki, and mixtures thereof.

The at least one filler may be present in an amount, when present, ranging from 0.001% to 35%, for instance from 0.5% to 15% by weight, relative to the total weight of the composition.

The at least one filler may be, for example, a filler with a mean particle size of less than 100 μm, for example ranging from 1 μm to 50 μm, such as from 4 μm to 20 μm.

According to one embodiment, the at least one first or at least one second composition according to the present disclosure comprises at least one filler that is present in an amount ranging from 0.01% to 60% by weight, relative to the total weight of the composition, for instance from 0.5% to 20%, such as from 1% to 10% by weight, relative to the total weight of the composition.

The at least one first and/or at least one second composition of the present disclosure may also comprise at least one cosmetic and/or dermatological active agents such as those conventionally used.

Among the cosmetic, dermatological, hygiene or pharmaceutical active agents that may be used in the composition of the present disclosure, non-limiting mention may be made of moisturizers, vitamins such as vitamin B3 and E, essential fatty acids, sphingolipids and sunscreens. These active agents are used in a usual amount for those skilled in the art, for example in an amount ranging from 0% to 20%, such as from 0.001% to 15% by weight, relative to the total weight of the at least one first or at least one second composition.

The compositions may also comprise any other additive usually used in such compositions, such as water, surfactants as described in French Patent Application No. FR 2 834 452, the content of which is incorporated into the present application by reference, antioxidants, fragrances, preserving agents and essential oils.

Needless to say, a person skilled in the art will take care to select this or these optional additional compound(s), and/or the amount thereof, such that the beneficial properties of the composition are not, or are not substantially, adversely affected by the envisaged addition.

The compositions of the product may be in the form of a cast product, for example in the form of a stick or wand, or in the form of a dish that may be used by direct contact or with a sponge. For instance, they can find an application as cast foundations, cast makeup rouges, cast eyeshadows, lipsticks, lipcare bases or balms, concealer products and nail varnishes. They may also be in the form of a soft paste or alternatively a gel, a more or less fluid cream, or a liquid packaged in a tube.

The compositions of the product according to the present disclosure may also be in the form of a cosmetic care composition for the face, the neck, the hands or the body (for example a care cream, an antisun oil or a body gel), a makeup composition (for example a makeup gel, a cream or a stick) or a composition for artificially tanning or for protecting the skin.

The compositions of the product according to the present disclosure may also be in the form of a care composition for the skin and/or the integuments or in the form of an antisun composition or a body hygiene composition, such as in the form of a deodorant. In such a case, the compositions may be for example, in uncolored form. They may then be used as a care base for the skin, the integuments or the lips (lip balms, for protecting the lips against the cold and/or sunlight and/or the wind, or care cream for the skin, the nails or the hair).

As used herein, the term “cosmetically acceptable” is understood to mean a composition of pleasant appearance, odour and feel.

Each composition of the product according to the present disclosure may be in any galenical form normally used for topical application, such as in the form of an oily or aqueous solution, an oily or aqueous gel, an oil-in-water or water-in-oil emulsion, a multiple emulsion, a dispersion of oil in water by means of vesicles, the vesicles being located at the oil/water interface, or a powder. Each composition may be fluid or solid.

According to one embodiment, the at least one first and/or at least one second composition can have a continuous fatty phase and can be in anhydrous form, comprising less than 5% water, such as less than 1% water, relative to the total weight of the at least one first and/or at least one second composition.

The at least one first and/or at least one second composition may have the appearance of a lotion, a cream, a pomade, a soft paste, an ointment, a cast or moulded solid, such as in stick or dish form, or alternatively a compacted solid.

Each composition may be packaged separately in the same packaging article, for example in a two-compartment pen, the base composition being delivered from one end of the pen and the top composition being delivered from the other end of the pen, each end being closed for example, in a leak-tight manner by a cap.

Alternatively, each of the compositions may be packaged in a different packaging article.

In one embodiment, the composition that is applied as first coat is in liquid or pasty form, for example, a lipstick or an eyeliner.

The product according to the present disclosure can be used, for example, for making up the skin and/or the lips and/or the integuments according to the nature of the ingredients used. For instance, the product of the present disclosure may be in the form of a solid foundation, a lipstick wand or paste, a concealer product, an eye contour product, an eyeliner, a mascara, an eyeshadow, a body makeup product or a skin coloring product.

For example the second composition can have care, gloss or transparency properties.

The present disclosure also relates to a lip product, a varnish, a mascara, a foundation, a tattoo, a makeup rouge or an eyeshadow comprising at least one first and at least one second composition as described above.

The compositions of the product of the present disclosure may be obtained by heating the various constituents to the melting point of the highest-melting waxes, followed by pouring the molten mixture into a mold (dish or finger stall). They may also be obtained by extrusion as described in European Patent Application No. EP-A-0 667 146.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The examples that follow are intended to illustrate the present disclosure in a non-limiting manner. The amounts are given as percentages by mass.

### EXAMPLES

### Example 1

First composition: Lipstick

| | % by |
|---|---|
| Ingredients | weight |
| Polyethylene wax | 10.5% |
| (weight-average molecular mass 500) | |
| Linear fatty alcohols | 2.5% |
| (Performacol 550 Alcohol sold by New Phase Technologies) | |
| Dispersion of methyl acrylate/acrylic acid copolymer | 68% |
| Sucrose acetate isobutyrate | 5% |
| (Eastman SAIB sold by Eastman Chemical) | |
| Pigmentary paste | 13.5% |
| Fragrance | 0.5% |

Preparation of the Copolymer Dispersion

A dispersion of non-crosslinked copolymer of methyl acrylate and of acrylic acid in a 95/5 ratio, in isododecane, was prepared according to the method of Example 1 of European Patent No. EP-A-749 746, replacing the heptane with isododecane. A dispersion of poly(methyl acrylate/acrylic acid) particles surface-stabilized in isododecane with a polystyrene/copoly(ethylene-propylene) diblock copolymer sold under the name Kraton G1701, with a solids content of 25% by weight, was thus obtained.

Preparation of the Lipstick

The polyethylene wax, the C30-50 alcohols and the pigmentary paste were introduced into a heating pan and heated to 100° C. with magnetic stirring, to obtain a homogeneous mixture. The pigmentary paste comprises 70% pigments, 1% poly(12-hydroxystearic acid) stearate and 29% hydrogenated polyisobutene. The sucrose acetate isobutyrate and the dispersion according to Example 1 were then added while maintaining the temperature and the stirring. The composition was poured into molds at 40° C., which were placed at −20° C. for 30 minutes. The sticks were then removed from the moulds. The sticks obtained had a homogeneous shade and were slippery on application. They gave a deposit on the lips that had good staying power, did not migrate, did not transfer and was not tacky.

Second Composition: Lip balm

| | % by |
|---|---|
| | Weight |
| Phenyl trimethicone (DC556 from Dow Corning) | 25 |
| Polydimethylsiloxane (Belsil 1000) | 25% |
| Polyglyceryl-3 polydimethylsiloxyethyl dimethicone (KF6104 ® | 12% |
| from Shin-Etsu) | |
| Polydimethylsiloxane (DC200, 10 cSt) | 15% |
| Octyldodecyl neopentanoate | 15% |
| Hydrophobic silica (Aerosil R972) | 8% |

### Example 2

First Composition: Lipstick

A lipstick comprising the polyglyceryl-3 polydimethylsiloxyethyl dimethicone sold under the reference KF6104® by Shin-Etsu, and whose composition is detailed in Table I below, was prepared.

TABLE I

| Ingredients | % by Weight |
|---|---|
| Hydrogenated polyisobutene (Parleam HV from NOF) | 4 |
| Isononyl isononanoate | 12 |
| 2-Octyldodecanol | 4.5 |
| Diisostearyl malate | 33.9 |
| 50/20/10/10 lauric/palmitic/cetylic/stearic acid triglycerides | 4 |
| (Softisan 100 ® from Sasol) | |
| N-Lauroyl-L-lysine | 1 |
| Copolymer of acrylates/stearyl acrylate/dimethicone | 4 |
| methacrylate (KP 561 P ® from Shin-Etsu) | |
| Dimethicone 6 cSt (KF 96) | 4 |
| Polyglyceryl-3 polydimethylsiloxyethyl dimethicone | 13 |
| (KF6104 ® from Shin-Etsu) | |
| Preserving agent | qs |
| Polyethylene wax (MW 500) | 6.6 |
| Polyethylene wax (MW 400) | 3.8 |
| Hydrophobic fumed silica surface-treated with | 2 |
| dimethylsilane (Aerosil R 972 ® from Degussa) | |
| Pigments | 7.0 |
| Simethicone (Antifoam C ® from Dow Corning) | 0.2 |
| Total | 100 |

Procedure

An oily phase was prepared by mixing together with heating (about 95° C.) the isononyl isononanoate, the 2-octyldodecanol and the diisostearyl malate with the polyglyceryl-3 polydimethylsiloxyethyl dimethicone and the dimethicone oil. The oily phase thus prepared was stirred at about 95° C. and the fillers (N-lauroyl-L-lysine and fumed silica) were added to the mixture.

The waxes, the pigments in the form of a pigmentary paste, the hydrogenated polyisobutene and the simethicone were then added to the mixture.

Second composition: Colorless wand of lipstick

| | % by |
|---|---|
| Ingredients | weight |
| Silicone oil (PDMS) | 25 |
| DC200 from Dow Corning (5 cSt) | |
| Dimethicone (and) Dimethiconol | 61 |
| D2-9085 from Dow Corning (1550 cSt) | |
| Trifluoropropyl dimethicone (100 cSt) | 1 |
| X22-819 from Shin-Etsu | |
| C30-45 alkyl dimethicone | 5 |
| (SF 1642 from GE Bayer Silicone) | |
| Polyethylene wax (weight-average MW 500) | 8 |

The silicone oil, the dimethiconol and the fluoro dimethicone were mixed together with heating until a homogeneous mixture was formed. The C30-C45 alkyl dimethicone was then added to the preceding mixture brought to 110° C. The polyethylene wax was then added portionwise until a homogeneous mixture was obtained. The mixture was cooled to 90-95° C. and then poured into molds, which were placed at −20° C. for 30 minutes. The sticks were finally removed from the molds.

### Example 3

First composition: Lipstick wand

| | % by |
|---|---|
| Ingredients | weight |
| Pigments | 8.20 |
| Hydrogenated polyisobutene (Parleam) | 5.18 |
| Polyhydroxystearic acid (Octacare DSPOL300, Avecia) | 0.21 |
| C30-C50 alcohols (Performacol 550 Alcohol, New Phase | 2 |
| Technologies) | |
| Polyethylene (Polywax 500, Bareco) | 10 |
| Sucrose acetate isobutyrate (Eastman SAIB Special, Eastman | 5 |
| Chemical) | |
| Grafted methyl acrylate polymer | 68.82 |
| Fragrance | qs |

Preparation of the Polymer Obtained by Polymerization of Methyl Acrylate and of the Macromonomer Corresponding to a Polyethylene/Polybutylene Copolymer Containing a Methacrylate End Group (Kraton L-1253)

2 kg of heptane, 2 kg of isododecane, 2.8 kg of methyl acrylate and 1.2 kg of macromonomer such as the copolymer of polyethylene/polybutylene type comprising a methacrylate end group (Kraton L-1253) and 320 g of tert-butyl peroxy-2-ethylhexanoate (Trigonox 21 S) were placed in a reactor.

The reaction mixture was stirred and heated from room temperature to 90° C. over 1 hour. After 15 minutes at 90° C., a change was observed in the appearance of the reaction medium, which changes from a transparent appearance to a milky appearance. Heating was continued with stirring for a further 15 minutes and a mixture comprising of 16 kg of methyl acrylate and 200 g of Trigonox 21 S was then added dropwise over 1 hour.

The mixture was heated for 4 hours at 90° C. and the heptane was then distilled from the reaction medium. After this distillation operation, a stable dispersion of polymer particles thus prepared in isododecane was obtained.

The grafted polymer comprises 6% by weight of macromonomer relative to the weight of the polymer.

The characteristics of the polymer and of the particles formed by the polymer were as follows:
weight-average molecular mass Mw=119,900
number-average molecular mass Mn=16,300
polydispersity index (Mw/Mn)=7.37
glass transition: 10° C. by Mettler DSC
dry extract: 52.4% in isododecane, performed by thermobalance
particle size: 46 nm with polydispersity of 0.05 performed on a Malvern Lo-C Autosizer at 25° C.
   Preparation of the Lipstick

The waxes, the pigmentary pastes and the sucrose ester were placed in a heating pan, with stirring using a Rayneri blender, the system was heated to 105° C. and left stirring for 30 minutes, the nacres were then added, followed by addition of the polymer dispersion and the fragrance, and the mixture was stirred for 10 minutes and then poured into a mold at 42° C. The mould was placed in a freezer and the lipstick was removed from the mold when the mold was at 4° C.

Second composition: Lip balm

| | % by |
|---|---|
| Ingredients | weight |
| Polyethylene wax | 2 |
| Ozokerite wax | 3 |
| Beeswax | 3 |
| Microcrystalline wax | 2 |
| Paraffin wax (EMW-3 from the company Nippon Seiro) | 8 |
| Sucrose polycottonseedate | 5 |
| Hydrogenated polyisobutene | 10 |
| Diisostearyl malate | 10 |
| Neopentyl glycol dicaprate | 10 |
| Isotridecyl isononanoate | 10 |
| Copolymer of acrylates/stearyl acrylate/dimethicone | 2 |
| methacrylate (KP 561 P ® from Shin-Etsu) | |
| Polyglyceryl-3 polydimethylsiloxyethyl dimethicone (KF6104 ® | 10 |
| from Shin-Etsu) | |
| Trimethyl siloxysilicate (SR 1000 ®) | 2 |
| Dimethicone 6 cSt | 5 |
| Titanium-coated mica | 5 |
| Reflective pigment | 2 |
| (Metashine ® from Nippon Sheet Glass) | |
| Titanium dioxide | 1 |

What is claimed is:

1. A cosmetic product comprising at least one first and at least one second composition, wherein
the at least one first composition comprises at least one silicone polymer of formula (I):
R¹ₐR²_{b}R³_{c}SiO_{(4-a-b-c)/2} (I)
wherein:
a ranges from 1 to 2.5,
b and c, independently of each other, range from 0.001 to 1.5;
R¹, which may be identical or different, is chosen from:
C₁ to C₃₀ alkyl radicals, optionally substituted with at least one entity chosen from fluorine atoms and amino and/or carboxyl groups,
aryl and aralkyl radicals, and
the radicals of formula (II):
—C_{d}H_{2d}—O—(C₂H₄O)ₑ(C₃H₆₀)_{f}R⁴ (II)
wherein:
R⁴ is chosen from C₁ to C₃₀ hydrocarbon-based radicals and R⁵—(CO)— radicals in which R⁵ is chosen from C₁ to C₃₀ hydrocarbon-based radicals, and
d, e and f are integers such that
d ranges from 0 to 15, and
e and f, independently of each other, range from 0 to 50,
and combinations thereof,
R² is a radical chosen from those of formula (III):
-Q-O—X (III)
wherein:
Q is a divalent radical chosen from C₂ to C₂₀ hydrocarbon-based radicals that may comprise at least one ether bond and/or at least one ester bond, and
X is a polyhydroxylated hydrocarbon-based radical,
R³ is a radical chosen from:
C₁ to C₃₀ alkyl radicals, optionally substituted with at least one entity chosen from fluorine atoms and amino and/or carboxyl groups,
aryl and aralkyl radicals,
organosiloxane groups of formula (IV): 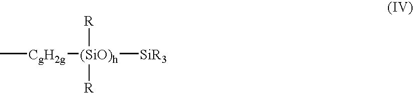
wherein:
R, which may be identical or different, are chosen from C₁ to C₃₀ alkyl radicals, optionally substituted with at least one entity chosen from fluorine atoms, and aryl and aralkyl radicals,
g and h are integers such that
g ranges from 1 to 5 and
h ranges from 0 to 500;
and the at least one second composition is different from the at least one first composition and comprises a cosmetically acceptable medium.

2. The cosmetic product according to claim 1, wherein R₁ is a C₁ to C₁₀ alkyl radical.

3. The cosmetic product according to claim 2, wherein R₁ is a C₁ to C₄, alkyl radical.

4. The cosmetic product according to claim 1, wherein:
a ranges from 1 to 1.4,
b and c, independently of each other, range from 0.02 to 0.04, and
R¹ is a C₁ to C₁₀ alkyl radical,
R² is a radical chosen from those of formula (IIIA):
—C₃H₆O[CH₂CH(OH)CH₂O]ₙH (IIIA)
in which n ranges from 1 to 5, and
R³ is a radical chosen from those of formula (IVA):
—C₂H₄(CH₃)₂SiO[(CH₃)₂SiO]ₘSi(CH₃)₂ (IVA)
in which m ranges from 3 to 9.

5. The cosmetic product according to claim 4, wherein R₁ is a C₁ to C₄, alkyl radical.

6. The cosmetic product according to claim 4, wherein the at least one silicone polymer is a compound of formula (I):
R¹ₐR²_{b}R³_{c}SiO_{(4-a-b-c)/2} (I)
wherein:
a ranges from 1 to 1.4,
b and c, independently of each other, range from 0.02 to 0.04,
R¹ is a methyl radical,
R² is a radical chosen from those of formula (IIIA) in which n ranges from 1 to 5, and
R³ is a radical chosen from those of formula (IVA) in which m ranges from 3 to 9.

7. The cosmetic product according to claim 1, wherein Q is a divalent radical chosen from —(CH₂)₂— and —(CH₂)₃— radicals.

8. The cosmetic product according to claim 1, wherein X is a polyhydroxylated hydrocarbon-based radical comprising at least two hydroxyl residues.

9. The cosmetic product according to claim 8, wherein X is a hydrocarbon-based group chosen from glycerol derivatives and saccharide derivatives.

10. The cosmetic product according to claim 1, wherein Q-O—X is a radical chosen from: 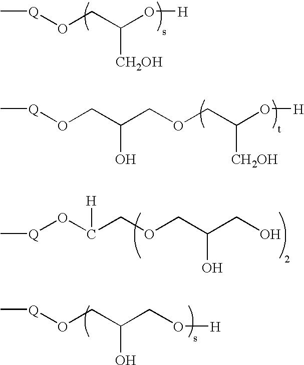
wherein s and t are integers ranging from 1 to 20.

11. The cosmetic product according to claim 10, wherein s and t are integers ranging from 1 to 5.

12. The cosmetic product according to claim 1, wherein the at least one silicone polymer is a polyglyceryl-3 polymethylsiloxyethyl dimethicone of formula (V): 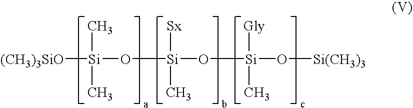
wherein:
—Sx is chosen from —C₂H₄[(CH₃)₂SiO]ₘSi(CH₃)₃ radicals, and
Gly is chosen from —C₃H₆O[CH₂—CH(OH)CH₂O]ₙH radicals,
in which a ranges from 1 to 1.4, b ranges from 0.02 to 0.04, c ranges from 0.02 to 0.04, m ranges from 3 to 9, and n ranges from 1 to 5.

13. The cosmetic product according to claim 1, wherein the at least one silicone polymer is a lauryl polyglyceryl-3 polymethylsiloxyethyl dimethicone of formula (VI): 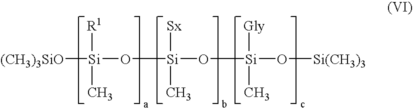
wherein
Sx is chosen from —C₂H₄[(CH₃)₂SiO]ₘSi(CH₃)₃ radicals, and
Gly is chosen from —C₃H₆O[CH₂—CH(OH)CH₂O]ₙH radicals,
in which a ranges from 1 to 1.4, b ranges from 0.02 to 0.04, c ranges from 0.02 to 0.04, m ranges from 3 to 9, and n ranges from 1 to 5, and
R¹ is chosen from methyl and lauryl radicals.

14. The cosmetic product according to claim 1, wherein the at least one silicone polymer is a polyglyceryl-3 disiloxane dimethicone (formula (VII)): 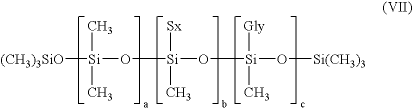
wherein
Sx is chosen from —O(CH₃)₂SiO—Si(CH₃)₃ radicals,
Gly is chosen from —C₃H₆O[CH₂—CH(OH)CH₂O]ₙH radicals,
in which a ranges from 1 to 1.4, b ranges from 0.02 to 0.04, c ranges from 0.02 to 0.04, m ranges from 3 to 9, and n ranges from 1 to 5.

15. The cosmetic product according to claim 1, wherein the at least one silicone polymer of formula (I) is present in the at least one first composition in an amount ranging from 0.1% to 40% by weight, relative to the total weight of the first composition.

16. The cosmetic product according to claim 15, wherein the at least one silicone polymer of formula (I) is present in the at least one first composition in an amount ranging from 7% to 15% by weight, relative to the total weight of the first composition.

17. The cosmetic product according to claim 1, wherein the at least one silicone polymer is such that when present in the at least one first composition in a sufficient amount, the product forms a deposit that has a transfer value ΔE (T) ranging from 0 to 45

18. The cosmetic product according to claim 17, wherein the transfer value ΔE (T) is less than or equal to 35.

19. The cosmetic product according to claim 1, wherein the at least one silicone polymer is such that when present in the at least one first composition in a sufficient amount, the composition forms a deposit that has a color fastness to water ΔE (E) of less than or equal to 15.

20. The cosmetic product according to claim 19, wherein the color fastness to water ΔE (E) of the composition is less than or equal to 6.

21. The cosmetic product according to claim 1, wherein the at least one silicone polymer is such that when present in the at least one first composition in a sufficient amount, the composition forms a deposit that has a color fastness to oil ΔE (H) of less than or equal to 25.

22. The cosmetic product according to claim 1, wherein the composition has a color fastness to oil ΔE (H) of less than or equal to 8.

23. The cosmetic product according to claim 1, wherein the at least one silicone polymer is such that when present the at least one first composition in a sufficient amount, the composition forms a deposit that has a mean gloss of greater than or equal to 30.

24. The cosmetic product according to claim 1, wherein the deposit that has a mean gloss of greater than or equal to 45.

25. The cosmetic product according to claim 1, wherein the at least one silicone polymer of formula (I) is such that when present in the at least one first cosmetic composition in a sufficient amount, the comfort index of a deposit of the product, when applied to a support, is greater than or equal to 80 over 100.

26. The cosmetic product according to claim 1, wherein the comfort index of a deposit of the product, when applied to a support, is greater than or equal to 95 over 100.

27. The cosmetic product according to claim 1, wherein the at least one first and the at least one second composition comprise, independently of each other, at least one non-volatile silicone oil chosen from non-volatile polydimethylsiloxanes (PDMS) optionally comprising alkyl or alkoxy groups that are pendent and/or at the end of a silicone chain, these groups each comprising from 2 to 24 carbon atoms, and phenyl silicones.

28. The cosmetic product according to claim 1, wherein the at least one first and/or at least one second composition comprises, independently of each other, at least one hydrocarbon-based oil chosen from hydrocarbon-based esters comprising less than 40 carbon atoms, of formula RCOOR′ in which RCOO is chosen from fatty acid residues comprising from 2 to 28 carbon atoms, and R′ is chosen from hydrocarbon-based chains comprising from 1 to 28 carbon atoms.

29. The cosmetic product according to claim 1, wherein the at least one first composition comprises at least one hydrocarbon-based oil chosen from hydrogenated polyisobutene, isostearyl heptanoate, isononyl isononanoate, isotridecyl isononanoate, diisostearyl malate, dipentaerythrityl tetrahydroxystearate/tetraisostearate and 2-octyldodecanol.

30. The cosmetic product according to claim 1, wherein the at least one first composition comprises at least one film-forming silicone polymer different from the at least one silicone polymer of formula (I).

31. The cosmetic product according to claim 30, wherein the at least one film-forming silicone polymer is chosen from siloxysilicates, polysilsesquioxanes and polymethylsesquioxanes.

32. The cosmetic product according to claim 30, wherein the at least one film-forming silicone polymer is chosen from copolymers comprising carboxylate groups and polydimethylsiloxane groups.

33. The cosmetic product according to claim 32, wherein the at least one film-forming silicone polymer is chosen from copolymers of acrylic acid and of stearyl acrylate comprising polydimethylsiloxane grafts, copolymers of stearyl methacrylate comprising polydimethylsiloxane grafts, copolymers of acrylic acid and of stearyl methacrylate comprising polydimethylsiloxane grafts, and copolymers of methyl methacrylate, butyl methacrylate, 2-ethylhexyl acrylate and stearyl methacrylate comprising polydimethylsiloxane grafts.

34. The cosmetic product according to claims 1, wherein the at least one first and/or at least one second composition comprises at least one wax and at least one pasty compound.

35. The cosmetic product according to claim 1 wherein the at least one second composition is transparent.

36. The cosmetic product according to claim 1, wherein the at least one second composition comprises at least one pigment, wherein the at least one pigment is present in an amount of less than 5% by weight, relative to the total weight of the at least one second composition.

37. The cosmetic product according to claim 36, wherein the at least one pigment is present in an amount of less than 1% by weight, relative to the total weight of the at least one second composition.

38. The cosmetic product according to claim 1, wherein the at least one second composition is chosen so as to improve at least one cosmetic property of the at least one first composition.

39. The cosmetic product according to claim 1, wherein the at least one first composition comprises at least one coloring agent chosen from liposoluble dyes, water-soluble dyes, pigments and nacres.

40. The cosmetic product according to claim 1, wherein the at least one first and/or at least one second composition is in the form of an anhydrous liquid, an anhydrous stick or an emulsion.

41. The cosmetic product according to claim 1, wherein it is in a form chosen from a foundation, a makeup rouge, an eyeshadow, a lipstick, a product with care properties, a mascara, an eyeliner, a nail varnish, a concealer product or a body makeup product.

42. A cosmetic process for making up the skin and/or the lips and/or the integuments, comprising applying to the skin, the lips and/or the integuments at least one first coat of at least one first composition comprising at least one coloring agent and at least one silicone polymer of formula (I):
R¹ₐR²_{b}R³_{c}SiO_{(4-a-b-c)/2} (I)
wherein:
a ranges from 1 to 2.5,
b and c, independently of each other, range from 0.001 to 1.5;
R¹, which may be identical or different, is chosen from:
C₁ to C₃₀ alkyl radicals, optionally substituted with at least one entity chosen from fluorine atoms and amino and/or carboxyl groups,
aryl and aralkyl radicals, and
the radicals of formula (II):
—C_{d}H_{2d}—O—(C₂H₄O)ₑ(C₃H₆O)_{f}R⁴ (II)
wherein:
R⁴ is chosen from C₁ to C₃₀ hydrocarbon-based radicals and R⁵—(CO)— radicals in which R⁵ is chosen from C₁ to C₃₀ hydrocarbon-based radicals, and
d, e and f are integers such that
d ranges from 0 to 15, and
e and f, independently of each other, range from 0 to 50,
and combinations thereof,
R² is a radical chosen from those of formula (III):
-Q-O—X (III)
wherein:
Q is a divalent radical chosen from C₂ to C₂₀ hydrocarbon-based radicals that may comprise at least one ether bond and/or at least one ester bond, and
X is a polyhydroxylated hydrocarbon-based radical,
R³ is a radical chosen from:
C₁ to C₃₀ alkyl radicals, optionally substituted with at least one entity chosen from fluorine atoms and amino and/or carboxyl groups,
aryl and aralkyl radicals,
organosiloxane groups of formula (IV): 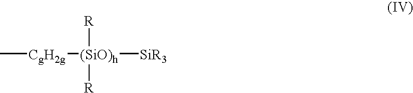
wherein:
R, which may be identical or different, are chosen from C₁ to C₃₀ alkyl radicals, optionally substituted with at least one entity chosen from fluorine atoms, and aryl and aralkyl radicals,
g and h are integers such that
g ranges from 1 to 5 and
h ranges from 0 to 500;
and then applying, onto all or part of the at least one first coat, at least one second coat of at least one second composition comprising a cosmetically acceptable medium.

43. A makeup kit comprising a cosmetic product comprising at least one first and at least one second composition, wherein
the at least one first composition comprises at least one silicone polymer of formula (I):
R¹ₐR²_{b}R³_{c}SiO_{(4-a-b-c)/2} (I)
wherein:
a ranges from 1 to 2.5,
b and c, independently of each other, range from 0.001 to 1.5;
R¹, which may be identical or different, is chosen from:
C₁ to C₃₀ alkyl radicals, optionally substituted with at least one entity chosen from fluorine atoms and amino and/or carboxyl groups,
aryl and aralkyl radicals, and
the radicals of formula (II):
—C_{d}H_{2d}—O—(C₂H₄O)ₑ(C₃H₆O)_{f}R⁴ (II)
wherein:
R⁴ is chosen from C₁ to C₃₀ hydrocarbon-based radicals and R⁵—(CO)— radicals in which R⁵ is chosen from C₁ to C₃₀ hydrocarbon-based radicals, and
d, e and f are integers such that
d ranges from 0 to 15, and
e and f, independently of each other, range from 0 to 50,
and combinations thereof,
R² is a radical chosen from those of formula (III):
-Q-O—X (III)
wherein:
Q is a divalent radical chosen from C₂ to C₂₀ hydrocarbon-based radicals that may comprise at least one ether bond and/or at least one ester bond, and
X is a polyhydroxylated hydrocarbon-based radical,
R³ is a radical chosen from:
C₁ to C₃₀ alkyl radicals, optionally substituted with at least one entity chosen from fluorine atoms and amino and/or carboxyl groups,
aryl and aralkyl radicals,
organosiloxane groups of formula (IV): 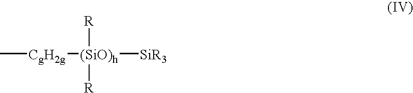
wherein:
R, which may be identical or different, are chosen from C₁ to C₃₀ alkyl radicals, optionally substituted with at least one entity chosen from fluorine atoms, and aryl and aralkyl radicals,
g and h are integers such that
g ranges from 1 to 5 and
h ranges from 0 to 500;
and the at least one second composition is different from the at least one first composition and comprises a cosmetically acceptable medium;
wherein each of the at least one first and at least one second compositions are packaged separately in at least one first and at least one second compartments.

44. The makeup kit according to claim 43, wherein the at least one first and at least one second compartments are formed inside the same container.

45. The makeup kit according to claim 43, wherein the at least one first and at least one second compartments are formed in two separate containers.

46. A method for providing a cosmetic product that is comfortable, glossy, transfer-resistant and/or migration-resistant and/or that has good color fastness and/or good gloss staying power, comprising preparing a cosmetic product comprising at least one first and at least one second compositions, wherein
the at least one first composition comprises at least one silicone polymer of formula (I):
R¹ₐR²_{b}R³_{c}SiO_{(4-a-b-c)/2} (I)
wherein:
a ranges from 1 to 2.5,
b and c, independently of each other, range from 0.001 to 1.5;
R¹, which may be identical or different, is chosen from:
C₁ to C₃₀ alkyl radicals, optionally substituted with at least one entity chosen from fluorine atoms and amino and/or carboxyl groups,
aryl and aralkyl radicals, and
the radicals of formula (II):
—C_{d}H_{2d}—O—(C₂H₄O)ₑ(C₃H₆O)_{f}R⁴ (II)
wherein:
R⁴ is chosen from C₁ to C₃₀ hydrocarbon-based radicals and R⁵—(CO)— radicals in which R⁵ is chosen from C₁ to C₃₀ hydrocarbon-based radicals, and
d, e and f are integers such that
d ranges from 0 to 15, and
e and f, independently of each other, range from 0 to 50,
and combinations thereof,
R is a radical chosen from those of formula (III):
-Q-O—X (III)
wherein:
Q is a divalent radical chosen from C₂ to C₂₀ hydrocarbon-based radicals that may comprise at least one ether bond and/or at least one ester bond, and
X is a polyhydroxylated hydrocarbon-based radical,
R³ is a radical chosen from:
C₁ to C₃₀ alkyl radicals, optionally substituted with at least one entity chosen from fluorine atoms and amino and/or carboxyl groups,
aryl and aralkyl radicals,
organosiloxane groups of formula (IV): 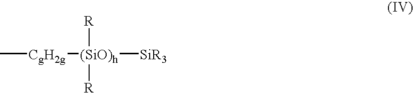
wherein:
R, which may be identical or different, are chosen from C₁ to C₃₀ alkyl radicals, optionally substituted with at least one entity chosen from fluorine atoms, and aryl and aralkyl radicals,
g and h are integers such that
g ranges from 1 to 5 and
h ranges from 0 to 500; and
the at least one second composition comprises a cosmetically acceptable medium,
wherein at least one of the at least one first and/or at least one second compositions comprise at least one coloring agent, and
further wherein the at least one first and at least one second composition are present in an amount sufficient to give the skin and/or the lips and/or the integuments a makeup that is comfortable, glossy, transfer-resistant and/or migration-resistant and/or that has good color fastness and/or good gloss staying power.
